(19) United States
(12) Patent Application Publication
(10) Pub. No.: US 2015/0166629 A1
(43) Pub. Date: Jun. 18, 2015

(54) SorCS1 FOR USE IN THE TREATMENT OF OBESITY AND OVERWEIGHT

(71) Applicant: Aarhus Universitet, Aarhus C (DK)

(72) Inventors: Karen-Marie Pedersen, Tjele (DK); Anders Nykjær, Risskov (DK); Mads Fuglsang Kjølby, Risskov (DK)

(21) Appl. No.: 14/390,937

(22) PCT Filed: Apr. 17, 2013
(86) PCT No.: PCT/DK2013/050107

§ 371 Date: Oct. 6, 2014

(30) Foreign Application Priority Data
Apr. 17, 2012 (DK) PA 2012 70191

(51) Int. Cl. C07K 14/705

(52) U.S. Cl. CPC C07K 14/705; A61K 38/00

(57) ABSTRACT

The present invention relates to SorCS1-like agents, including SorCS1, nucleic acid molecule encoding expression of SorCS1 and fragments thereof, as well as vectors containing said nucleic acid and to cells expressing SorCS1 and said fragments, for use in a method of reducing appetite, and/or for promoting weight loss, and/or for treating obesity, and/or for increasing metabolism, and/or for increasing thermogenesis, and/or for converting white fat into brown fat

The present application claims priority from Danish patent application no. PA 2012 70191, filed 17 Apr. 2012. All references cited in that application and in the present application are hereby incorporated by reference in their entirety.

### FIELD OF INVENTION

The present invention relates to a method of reducing appetite, suppressing hunger and/or treating obesity by administering SorCS1, preferably SorCS1 polypeptides and soluble fragments and variants thereof.

### BACKGROUND OF THE INVENTION

Obesity is a medical condition in which body fat has accumulated to an extent that it may have adverse effects on health. Clinically, obesity is defined by the World health Organization (WHO) as having a Body Mass Index (BMI) over 30. Within the obese population, three distinct sub-classes can be defined, based on the severity of obesity, ranging from class I obesity (BMI 30.0-34.9), class II obesity (BMI 35.0-39.9) and class III obesity (BMI over 40), which are also cumulative issues for public health action. It is estimated that up to 15% of all adults in Denmark suffer from obesity (BMI>30).

Adverse consequences of obesity are, a negative social image, cardiovascular disease and type 2 diabetes (Darvall et al., Eur J Vasc Endovasc Surg 2007, Haslam & James, Lancet 2005, Vernochet et al., FEBS J 2009, Yusuf et al., Lancet 2004), as well as several cancers (Roberts et al., Annu Rev Med 2009). In addition to these adverse effects, obesity is also associated with a number of other co-morbidities such as psychiatric- and neurological disorders (Beydoun et al., Obes Rev 2008, Harney et al., Pain Med 2007).

Currently, obesity is one of the most important risk factors attributing to disease-burden worldwide, and the second leading preventable cause of death (after smoking) in the US (Mokdad et al., JAMA 2004). In 2005, 1.1 billion adults and 10% of children were classified as overweight or obese (Haslam & James, Lancet 2005). In Europe, the incidence of obesity is increasing, and maybe of even more concern; childhood obesity is becoming more and more prevalent (Livingstone, Public Health Nutr 2011).

The current treatments of obesity include dietary changes, exercise and activity, behavior changes, prescription weight-loss medications and weight-loss surgery. Weight-loss drugs in sale and development include molecules intended to reduce the absorption from the gastro-intestinal (GI) tract (Orlistat), or various ways to limit food intake and suppress hunger (Phentermine, Pramlintide, Exenatide, Liraglutide). However, only Orlistat end Phentermine is approved for sale as weight loss drugs. Orlistat (Xenical) reduces intestinal fat absorption by inhibiting pancreatic lipase. Some side-effects of using Orlistat include frequent, oily bowel movements (steatorrhea). But if fat in the diet is reduced, symptoms often improve. Originally available only by prescription, it was approved by the FDA for over-the-counter sale in February 2007. Phentermine is a psychostimulant drug of the phenethylamine class, with pharmacology similar to amphetamine. It is approved as an appetite suppressant to help reduce weight in obese patients when used short-term and combined with exercise, diet, and behavioral modification. Pramlintide (Symlin) is a synthetic analogue of the hormone Amylin, which in normal people is secreted by the pancreas in response to eating. Among other effects, Amylin delays gastric emptying and promotes a feeling of satiety. Many diabetics are deficient in Amylin. Symlin is only approved to be used along with insulin by Type 1 and Type 2 diabetics. However, Symlin is currently being tested in non-diabetics as a treatment for obesity. Exenatide (Byetta) is a long-acting analogue of the hormone GLP-1, which the intestines secrete in response to the presence of food. Among other effects, GLP-1 delays gastric emptying and promotes a feeling of satiety. Some obese people are deficient in GLP-1, and dieting reduces GLP-1 further. Byetta is currently available as a treatment for type 2 diabetes. Some, but not all, patients find that they lose substantial weight when taking Byetta. However, Byetta is only approved and recommended for patients with Type 2 Diabetes. Liraglutide (Victoza) is a long-acting glucagon-like peptide-1 (GLP-1) analog. Among other effects, Victoza increase insulin secretion, delay gastric emptying, and suppress prandial glucagon secretion. Victoza is currently available as a treatment for type 2 diabetes. Some patients find that they lose substantial weight when taking Victoza. However, Victoza is only approved and recommended for patients with Type 2 Diabetes.

Weight loss surgery includes gastric bypass surgery, laparoscopic adjustable gastric banding (LAG B), gastric sleeve and biliopancreatic diversion with duodenal switch The Vps10p-domain (Vps10p-D) receptor family, comprises the receptors Sortilin, SorLA, SorCS1, SorCS2, and SorCS3. They are all type-1 transmembrane receptors sharing the characteristic structural feature of an N-terminal Vps10p-domain with high sequence identity to Vps10p, a sorting protein in yeast (10). Recent findings indicate that both Sortilin and SorLA play a crucial role as regulators of neuronal survival and death (11,12, WO 2004/056385, WO 2008/074329). Interestingly, Sortilin has also been associated with insulin-regulated glucose up-take as it may facilitate translocation of the glucose transporter GLUT4 from an intracellular compartment to the plasma membrane (13,14, WO 2010/142296).

SorCS1 is a receptor that, among other tissues, is expressed in the brain, pancreas, fatty tissue and muscles. Genetic studies have shown that polymorphisms in the SORCS1 gene in humans (Nat Genet. 2006 June; 38(6):688-93), rats (Genetics. 2006 November; 174(3):1565-72) and mice (Diabetes. 2007 July; 56(7):1922-9) are associated to risk of development of type-2 diabetes.

SorCS1 is unique among the Vps10p-D receptors as it exists in several distinct splice variants, denoted SorCS1-a, b, c, c+, and d, that encode identical extracellular and transmembrane parts, and cytoplasmic domains that differ in length and sequence (10, 11). It has been demonstrated that SorCS1, in addition to in the nervous system, is expressed in adipose tissue, skeletal muscle and (3-cells of the pancreas (WO 2010/142296).

It has also been demonstrated (WO 2010/142296) that SorCS1 can bind to the insulin receptor (IR) and stabilize its expression in muscle- and adipose tissue, hereby ensuring the ability to respond to insulin. To support this notion, treatment with the extracellular domain of SorCS1 (soluble SorCS1) results in a marked reduction in both plasma glucose and insulin levels in db/db mice (obesity dependent type-2 diabetic mice).

### SUMMARY OF THE INVENTION

SorCS1 is one of five members of the mammalian Vps10p-domain (Vps10p-D) receptor family, which also comprises Sortilin, SorLA, SorCS2, and SorCS3. SorCS1 is unique among the Vps10p-D receptors as it exists in several distinct splice variants.

The present inventors have found that administration of SorCS1, and in particular the extracellular domain of a SorCS1 polypeptide (soluble SorCS1 or sSorCS1) to a subject results in a significant weight reduction in the treated subjects.

It is thus an object of the present invention to provide methods and agents capable of reducing appetite, and/or suppressing hunger, and/or increasing the suppression of hunger, and/or increasing the reduction of prospective consumption and/or increasing the reduction of appetite, and/or increasing satiety, and/or treating obesity, and/or promoting weight loss, and/or increasing metabolism, and/or transforming white fat into brown fat. The latter results in increased thermogenesis of the subject receiving SorCS1 therapy. Thus the invention also concern a method of increasing thermogenesis in a subject.

Consequently in a main aspect the present invention relates to an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for reducing appetite, and/or for promoting weight loss, and/or treating obesity, and/or increasing metabolism, and/or increasing thermogenesis, and/or converting white fat into brown fat.

The agent of the invention may be formulated in a manner suitable for delivery to a subject. Thus in one aspect the invention concerns a pharmaceutical composition comprising the agent defined herein above. In one aspect the invention concerns a kit comprising said pharmaceutical composition, and instructions for use such as instructions for administration to a subject.

### DETAILED DESCRIPTION OF THE INVENTION

### Definitions

Unless specifically indicated otherwise, all technical and scientific terms used herein have the same meaning as commonly understood by those of ordinary skill in the art to which this invention belongs. For purposes of the present invention, the following terms are defined.

Acylation: The term “acylation” or “acylation group” as used herein means an R—(C═O)-group, wherein R is selected from straight-chain or branched, saturated or unsaturated carbon chains, optionally comprising one or more O, N, S, or P, such as a straight-chain or branched alkane carboxylic acid. Various examples of suitable acylation groups are described in WO2006/037810, WO00/34331, WO2006/097537, WO2011/080103. In particular examples of suitable acylation groups have the structure CH3(CH2)ₙCO—, wherein n is 4 to 40, e.g. 8 to 22, such as an acylation group selected from the group comprising:
CH₃(CH2)₈CO—, CH₃(CH₂)₉CO—, CH₃(CH₂)₁₀CO—, CH₃(CH₂)₁₁CO—, CH₃(CH₂)₁₂CO—, CH₃(CH₂)₁₃CO—, CH₃(CH₂)₁₄CO—, CH₃(CH₂)₁₅CO—, CH₃(CH₂)₁₆CO—, CH₃(CH₂)₁₇CO—, CH₃(CH₂)₁₈CO—, CH₃(CH₂)₁₉CO—, CH₃(CH₂)₂₀CO—, CH₃(CH₂)₂₁CO— and CH₃(CH₂)₂₂CO—. Further examples of suitable acylation groups has the structure HOOC—(CH₂)ₙCO—, wherein n is 4 to 40, e.g. 12 to 20, typically, HOOC—(CH₂)₁₄CO—, HOOC—(CH₂)₁₅CO—, HOOC—(CH₂)₁₆CO—, HOOC—(CH₂)₁₇CO— and HOOC—(CH₂)₁₈CO—. See also U.S. Pat. No. 5,905,140 for further examples of acylation groups.
Adjuvant: Any substance whose admixture with an administered immunogenic determinant/antigen increases or otherwise modifies the immune response to said determinant.
Affinity: The interaction of most ligands with their binding sites can be characterized in terms of a binding affinity. In general, high affinity ligand binding results from greater intermolecular force between the ligand and its receptor while low affinity ligand binding involves less intermolecular force between the ligand and its receptor. In general, high affinity binding involves a longer residence time for the ligand at its receptor binding site than is the case for low affinity binding. High affinity binding of ligands to receptors is often physiologically important when some of the binding energy can be used to cause a conformational change in the receptor, resulting in altered behavior of an associated ion channel or enzyme.

A ligand that can bind to a receptor, alter the function of the receptor and trigger a physiological response is called an agonist for that receptor. Agonist binding to a receptor can be characterized both in terms of how much physiological response can be triggered and the concentration of the agonist that is required to produce the physiological response. High affinity ligand binding implies that a relatively low concentration of a ligand is adequate to maximally occupy a ligand binding site and trigger a physiological response. Low affinity binding implies that a relatively high concentration of a ligand is required before the binding site is maximally occupied and the maximum physiological response to the ligand is achieved. Ligand binding is often characterized in terms of the concentration of ligand at which half of the receptor binding sites are occupied, known as the dissociation constant (k_{d}). Affinity is also the strength of binding between receptors and their ligands, for example between an antibody and its antigen.

Agonist: An agonist is a compound capable of increasing or effecting the activity of a receptor. Specifically, a Vps10p-domain receptor agonist is a compound capable of binding to one or more of binding sites of a Vps10p-domain receptor thereby inducing the same physiological response as a given endogenous agonist ligand compound.
Antagonist: An antagonist is in this case synonymous with an inhibitor. An antagonist is a compound capable of decreasing the activity of an effector such as a receptor. Specifically, a Vps10p-domain receptor antagonist is a compound capable of binding to one or more of binding sites of Vps10p-domain receptor thereby inhibiting binding of another ligand thus inhibiting a physiological response.
Antibody: The term “antibody” as referred to herein includes whole antibodies and any antigen binding fragment (i.e., “antigen-binding portion”) or single chain thereof.
Polyclonal antibody: Polyclonal antibodies are a mixture of antibody molecules recognising a specific given antigen, hence polyclonal antibodies may recognise different epitopes within said antigen.
Aromatic group: the term “aromatic group” or “aryl group” means a mono- or polycyclic aromatic hydrocarbon group.
Binding site: The term “binding site” or “binding pocket”, as used herein, refers to a region of a molecule or molecular complex that, as a result of its shape, favourably associates with another molecule, molecular complex, chemical entity or compound. As used herein, the pocket comprises at least a deep cavity and, optionally a shallow cavity.
Bioreactive agent or biologically active or biological activity: The terms as used herein refers to effect of any compound or substance which may be used in connection with an application that is therapeutic or otherwise useful according to this invention.
Electrostatic interaction: The term “electrostatic interaction” as used herein refers to any interaction occurring between charged components, molecules or ions, due to attractive forces when components of opposite electric charge are attracted to each other. Examples include, but are not limited to: ionic interactions, covalent interactions, interactions between a ion and a dipole (ion and polar molecule), interactions between two dipoles (partial charges of polar molecules), hydrogen bonds and London dispersion bonds (induced dipoles of polarizable molecules). Thus, for example, “ionic interaction” or “electrostatic interaction” refers to the attraction between a first, positively charged molecule and a second, negatively charged molecule. Ionic or electrostatic interactions include, for example, the attraction between a negatively charged bioactive agent.
Fc fragment: The term “an Fc fragment of a mammalian antibody” as used herein means a constant region, i.e. Fc fragment of a mammalian antibody or a fragment thereof wherein such mammalian antibody may be selected from IgM, IgG, IgA, IgD and IgE from a mammal, such as a primate, e.g. human, abe, or monkey; an equine, e.g. horse. A typical Fc fragment of a mammalian antibody is a recombinant Fc fragment of a human antibody, such as a recombinant Fc fragment of a human IgG antibody.

In the present context, the term “a variant of an Fc fragment of a mammalian antibody” or “Fc variant” (used interchangeably throughout the present description) as used herein means the Fc fragment of a mammalian antibody, wherein one or more amino acid residues, such as 1-10 amino acid residues, of the Fc fragment have been substituted by other amino acid residues and/or wherein one or more amino acid residues, such as 1-10 amino acid residues, have been deleted from the Fc fragment and/or wherein one or more amino acid residues, such as 1-10 amino acid residues, have been added to the Fc fragment and/or wherein one or more amino acid residues, such as 1-10 amino acid residues, in the Fc fragment have been modified. Such addition or deletion of amino acid residues can take e.g. place at the N-terminal of the Fc fragment and/or at the C-terminal of the Fc fragment. Native refers to an Fc that has not been modified by a human. WO 96/32478 describes exemplary Fc variants. Thus, the term “Fc variant” in one embodiment comprises a molecule or sequence that is humanized from a non-human native Fc. Furthermore, a native Fc comprises sites that may be removed because they provide structural features or biological activity that are not required for the fusion molecules of the present invention.

Fragments: The polypeptide fragments according to the present invention, including any functional equivalents thereof, may in one embodiment comprise less than 500 amino acid residues, such as less than 450 amino acid residues, for example less than 400 amino acid residues, such as less than 350 amino acid residues, for example less than 300 amino acid residues, for example less than 250 amino acid residues, such as less than 240 amino acid residues, for example less than 225 amino acid residues, such as less than 200 amino acid residues, for example less than 180 amino acid residues, such as less than 160 amino acid residues, for example less than 150 amino acid residues, such as less than 140 amino acid residues, for example less than 130 amino acid residues, such as less than 120 amino acid residues, for example less than 110 amino acid residues, such as less than 100 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues, such as less than 45 amino acid residues, for example less than 40 amino acid residues, such as 35 amino acid residues, for example 30 amino acid residues, such as 25 amino acid residues, such as 20 amino acid residues, for example 15 amino acid residues, such as 10 amino acid residues, for example 5 contiguous amino acid residues of an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64 or a variant thereof being at least 70% (e.g. at least 85%, 90%, 95%, 97%, 98%, or 99%) identical to said sequences. Also, the polypeptide fragments according to the present invention, including any functional equivalents thereof, may in one embodiment comprise more than 5 amino acid residues, such as more than 10 amino acid residues, for example more than 15 amino acid residues, such as more than 20 amino acid residues, for example more than 25 amino acid residues, for example more than 50 amino acid residues, such as more than 75 amino acid residues, for example more than 100 amino acid residues, such as more than 125 amino acid residues, for example more than 150 amino acid residues, such as more than 175 amino acid residues, for example more than 200 amino acid residues, such as more than 225 amino acid residues, for example more than 250 amino acid residues, such as more than 275 amino acid residues, for example more than 300 amino acid residues, such as more than 325 amino acid residues, for example more than 350 amino acid residues, such as more than 375 amino acid residues, for example more than 400 amino acid residues, such as more than 425 amino acid residues, for example more than 450 amino acid residues, such as more than 475 amino acid residues, for example more than 500 amino acid residues, such as more than 525 amino acid residues, for example more than 550 amino acid residues, such as more than 575 amino acid residues, for example more than 600 amino acid residues, such as 625 amino acid residues, for example 650 amino acid residues, such as 675 amino acid residues, such as 700 amino acid residues of an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64 or a variant thereof being at least 60% (e.g. at least 65%, 70%, 80%, 85%, 90%, 95%, 97%, 98%, or at least 99%) identical to said sequences. Examples of active fragments include one or more of the following: SEQ ID NO: 1 aa 103-124, SEQ ID NO: 1 aa 125-143, SEQ ID NO: 1 aa 144-162, SEQ ID NO: 1 aa 197-218, SEQ ID NO: 1 aa 391-409, SEQ ID NO: 1 aa 661-684, SEQ ID NO: 1 aa 763-783, or SEQ ID NO: 1 aa 859-876. The fragments may be from 5 to 500 amino acids in length, for example, 5 to 400, 10 to 300, 20 to 250, 15 to 50, 5 to 15, 7 to 15, 10 to 25, 10 to 20, and 7 to 25 amino acids in length.
Functional equivalency: “Functional equivalency” as used in the present invention is, according to one preferred embodiment, established by means of reference to the corresponding functionality of a predetermined fragment of the sequence.

Functional equivalents or variants of a SorCS1 polypeptide, or a fragment thereof will be understood to exhibit amino acid sequences gradually differing from the preferred predetermined SorCS1 polypeptide or the SorCS1 fragment sequence respectively, as the number and scope of insertions, deletions and substitutions including conservative substitutions increase, while retaining the biological activity of a SorCS1 polypeptide in this context. This difference is measured as a reduction in identity between the preferred predetermined sequence and the fragment or functional equivalent.

A functional variant obtained by substitution of one or more amino acid residues may well exhibit some form or degree of native SorCS1 activity, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity is not a principal measure of a fragment being a variant or functional equivalent of a preferred predetermined fragment according to the present invention.

In addition to conservative substitutions introduced into any position of a preferred predetermined SorCS1 polypeptide, or a fragment thereof, it may also be desirable to introduce non-conservative substitutions in any one or more positions of such a SorCS1 polypeptide, or a fragment thereof.

A non-conservative substitution leading to the formation of a functionally equivalent fragment of a SorCS1 polypeptide, or a fragment thereof would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Variants obtained by substitution of amino acids may in one preferred embodiment be made based upon the hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

Mutagenesis of a preferred predetermined SorCS1 polypeptide, or a fragment thereof, can be conducted by making amino acid insertions, usually on the order of about from 1 to 10 amino acid residues, preferably from about 1 to 5 amino acid residues, or deletions of from about from 1 to 10 residues, such as from about 2 to 5 residues.

In one embodiment the ligand of binding site 1, 2 or 3 is an oligopeptide synthesised by automated synthesis. Any of the commercially available solid-phase techniques may be employed, such as the Merrifield solid phase synthesis method, in which amino acids are sequentially added to a growing amino acid chain (see Merrifield, J. Am. Chem. Soc. 85:2149-2146, 1963).

Equipment for automated synthesis of polypeptides is commercially available from suppliers such as Applied Biosystems, Inc. of Foster City, Calif., and may generally be operated according to the manufacturer's instructions. Solid phase synthesis will enable the incorporation of desirable amino acid substitutions into any fragment of SorCS1 according to the present invention. It will be understood that substitutions, deletions, insertions or any subcombination thereof may be combined to arrive at a final sequence of a functional equivalent. Insertions shall be understood to include amino-terminal and/or carboxyl-terminal fusions, e.g. with a hydrophobic or immunogenic protein or a carrier such as any polypeptide or scaffold structure capable as serving as a carrier.

Oligomers including dimers including homodimers and heterodimers of fragments of sortilin inhibitors according to the invention are also provided and fall under the scope of the invention. SorCS1 polypeptides and fragments, functional equivalents and variants thereof can be produced as homodimers or heterodimers with other amino acid sequences or with native sortilin inhibitor sequences. Heterodimers include dimers containing immunoreactive sortilin inhibiting fragments as well as sortilin inhibiting fragments that need not have or exert any biological activity.

SorCS1 polypeptides, or fragments and variants thereof may be synthesised both in vitro and in vivo. Methods for in vitro synthesis are well known, and methods being suitable or suitably adaptable to the synthesis in vivo of sortilin inhibitors are also described in the prior art. When synthesized in vivo, a host cell is transformed with vectors containing DNA encoding a sortilin peptide inhibitor or a fragment thereof. A vector is defined as a replicable nucleic acid construct. Vectors are used to mediate expression of SorCS1 polypeptides, and/or fragments and variants. An expression vector is a replicable DNA construct in which a nucleic acid sequence encoding the predetermined sortilin inhibiting fragment, or any functional equivalent thereof that can be expressed in vivo, is operably linked to suitable control sequences capable of effecting the expression of the fragment or equivalent in a suitable host. Such control sequences are well known in the art. Both prokaryotic and eukaryotic cells may be used for synthesising ligands.

Cultures of cells derived from multicellular organisms however represent preferred host cells. In principle, any higher eukaryotic cell culture is workable, whether from vertebrate or invertebrate culture. Examples of useful host cell lines are VERO and HeLa cells, Chinese hamster ovary (CHO) cell lines, and W138, BHK, COS-7, 293 and MDCK cell lines. Preferred host cells are eukaryotic cells known to synthesize endogenous sortilin inhibitors. Cultures of such host cells may be isolated and used as a source of the fragment, or used in therapeutic methods of treatment, including therapeutic methods aimed at promoting or inhibiting a growth state, or diagnostic methods carried out on the human or animal body.

In vitro/in vivo: the terms are used in their normal meaning.
Ligand: a substance, compound or biomolecule such as a protein including receptors, that is able to bind to and form a complex with (a second) biomolecule to serve a biological purpose. In a narrower sense, it is a signal triggering molecule binding to a site on a target protein, by intermolecular forces such as ionic bonds, hydrogen bonds and Van der Waals forces. The docking (association) is usually reversible (dissociation). Actual irreversible covalent binding between a ligand and its target molecule is rare in biological systems. As opposed to the meaning in metalorganic and inorganic chemistry, it is irrelevant, whether or not the ligand actually binds at a metal site, as it is the case in hemoglobin. Ligand binding to receptors may alter the chemical conformation, i.e. the three dimensional shape of the receptor protein. The conformational state of a receptor protein determines the functional state of a receptor. The tendency or strength of binding is called affinity. Ligands include substrates, inhibitors, activators, non-self receptors, co-receptors and neurotransmitters.
Linker: The term “linker” as used herein means a valence bond or multifunctional moiety, such as a bifunctional moiety that separates the SorCS1 agent and the pharmaceutically acceptable molecule conjugated to SorCS1 and resulting in increased half-life such as increased plasma half-life.
Polymer: The term “polymer” as used herein means a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is intended to cover carbohydrate molecules attached by in vitro glycosylation. Carbohydrate molecules attached by in vivo glycosylation, such as N- or O-glycosylation (as further described below) are referred to herein as “an oligosaccharide moiety”. Except where the number of polymer molecules is expressly indicated, every reference to “a polymer”, “a polymer molecule”, “the polymer” or “the polymer molecule” as used in the present invention shall be a reference to one or more polymer molecule(s). The polymer may be a water soluble or water insoluble polymer, such as a PEG moiety. The PEG moiety may have an average size selected from the range of 500 Da to 200.000 Da, such as from 500 Da to 100.000 Da, such as from 2000 Da to 50.000 Da. Such PEG molecules may be retrieved from i.a. Shearwater Inc.
Pharmaceutical agent: The terms “pharmaceutical agent” or “drug” or “medicament” refer to any therapeutic or prophylactic use of an agent according to the invention, which agent may be used in the treatment (including the prevention, diagnosis, alleviation, or cure) of a malady, affliction, condition, disease or injury in a patient. Therapeutically useful genetic determinants, peptides, polypeptides and polynucleotides may be included within the meaning of the term pharmaceutical or drug. As defined herein, a “therapeutic agent”, “pharmaceutical agent” or “drug” or “medicament” is a type of bioactive agent.
Pharmaceutical composition: or drug, medicament or agent refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical composition” and “medicament” preferably encompass an active agent as such or an inactive drug and the active metabolite.
Purified antibody: The term a “purified antibody” is an antibody at least 60 weight percent of which is free from the polypeptides and naturally-occurring organic molecules with which it is naturally associated. Preferably, the preparation comprises antibody in an amount of at least 75 weight percent, more preferably at least 90 weight percent, and most preferably at least 99 weight percent.
Sequence identity: The term “sequence identity” or “identical” as used herein refers to a relationship between the sequences of two or more proteins, as determined by comparing the sequences. The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTP programs of Altschul, et al. (1990) J. Mol. Biol. 215:403-410.

In order to characterize the identity, subject sequences are aligned so that the highest order homology (match) is obtained. Based on these general principles, the “percent identity” of two nucleic acid sequences may be determined using the BLASTN algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Reward for a match=1; Penalty for a mismatch=−2; Strand option=both strands; Open gap=5; Extension gap=2; Penalties gap x_dropoff=50; Expect=10; Word size=11; Filter on). The BLASTN algorithm determines the % sequence identity in a range of overlap between two aligned nucleotide sequences.

Another preferred, non-limiting example of a mathematical algorithm utilized for the comparison of sequences is the CLUSTAL W (1.7) alignment algorithm (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680.). CLUSTAL W can be used for multiple sequence alignment preferably using BLOSUM 62 as scoring matrix. When calculating sequence identities, CLUSTAL W includes any gaps made by the alignment in the length of the reference sequence.

Sequence identities are calculated by dividing the number of Matches by the length of the aligned sequences with gaps.

A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence.

Treatment: The term “treatment” as used herein refers to a method involving therapy including surgery of a clinical condition in an individual including a human or animal body. The therapy may be ameliorating, curative or prophylactic, i.e. reducing mental and behavioural symptoms.
Variants: The term “variants” as used herein refers to amino acid sequence variants said variants preferably having at least 60% identity, for example at least 63% identity, such as at least 66% identity, for example at least 70% sequence identity, fear example at least 72% sequence identity, for example at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with any of the predetermined sequences.
Up-regulation of expression: a process leading to increased expression of genes, preferably of endogenous genes.

### DESCRIPTION OF THE DRAWINGS

FIG. 1: Alignment of SorCS1

Sequence alignment of SorCS1 from Human (homo sapiens), Chimpanzee (Pan troglodytes), Cow (Bos Taurus), Mouse (Mus musculus), Rat (Rattus norvegicus), Dog (Canis lupus familiaris) and Chicken (Gallus gallus) origin. The sequence identity is as demonstrated in table 2.

TABLE 2
Sequence identity to human SorCS1

| | Protein | DNA |
|---|---|---|
| Species | (% identity) | (% identity) |
| Human | 100 | 100 |
| Chimpanzee | 99.6 | 99.4 |
| Dog | 97.6 | 92.5 |
| Cow | 92.9 | 89.8 |
| Mouse | 93.2 | 87.7 |
| Rat | 93.2 | 88.0 |
| Chicken | 85.3 | 79.7 |

FIG. 2: Gene expression profiling of adipose tissue from SorCS1 knockout mice by PCR arrays.

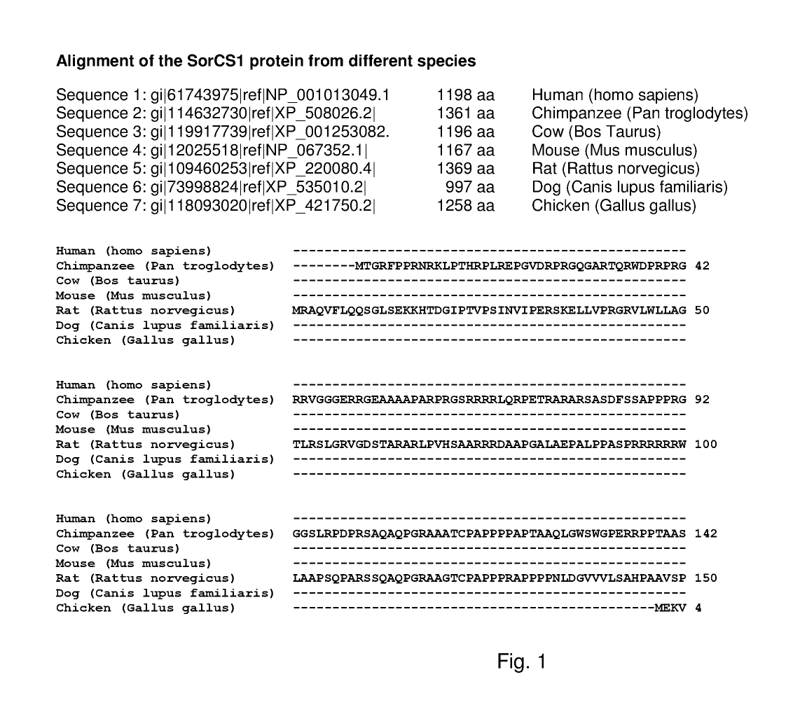

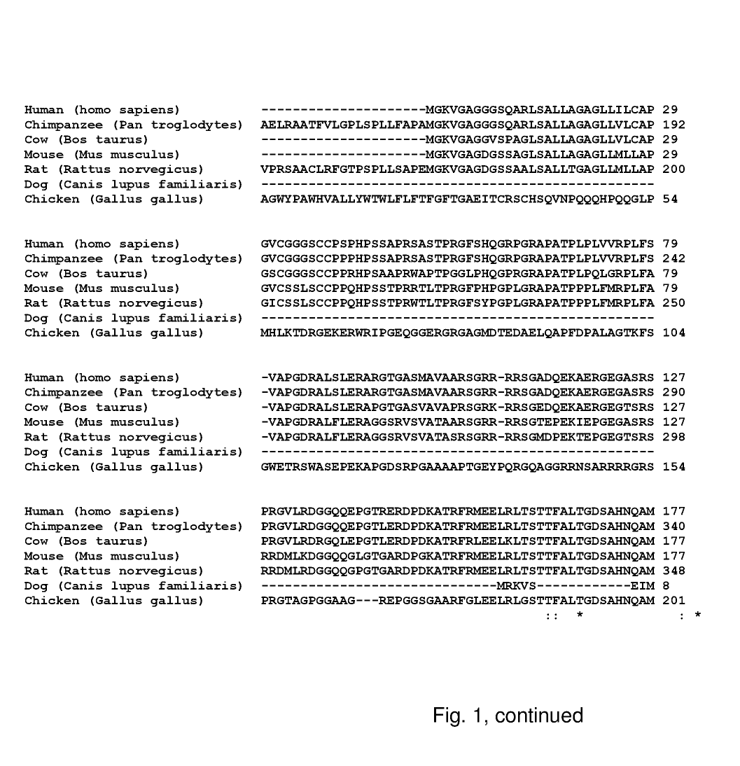

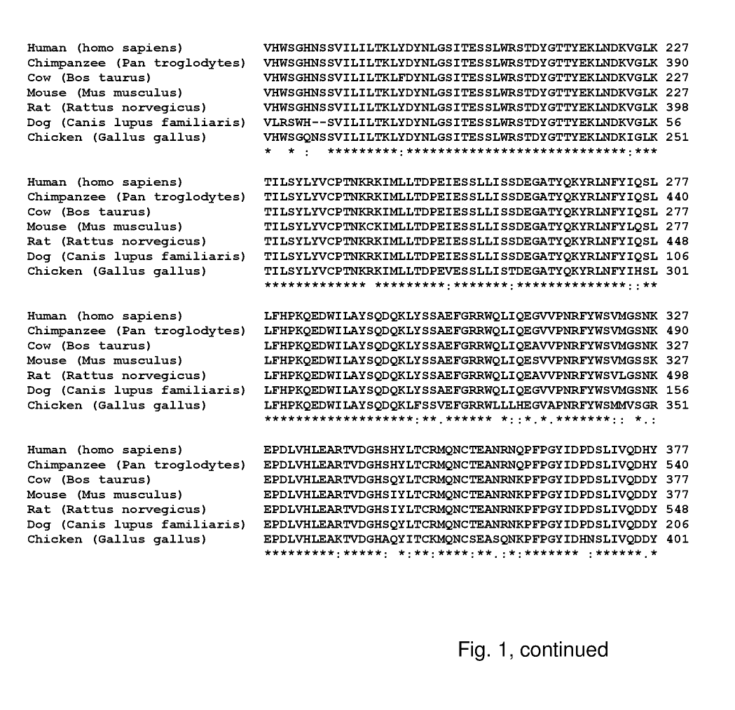

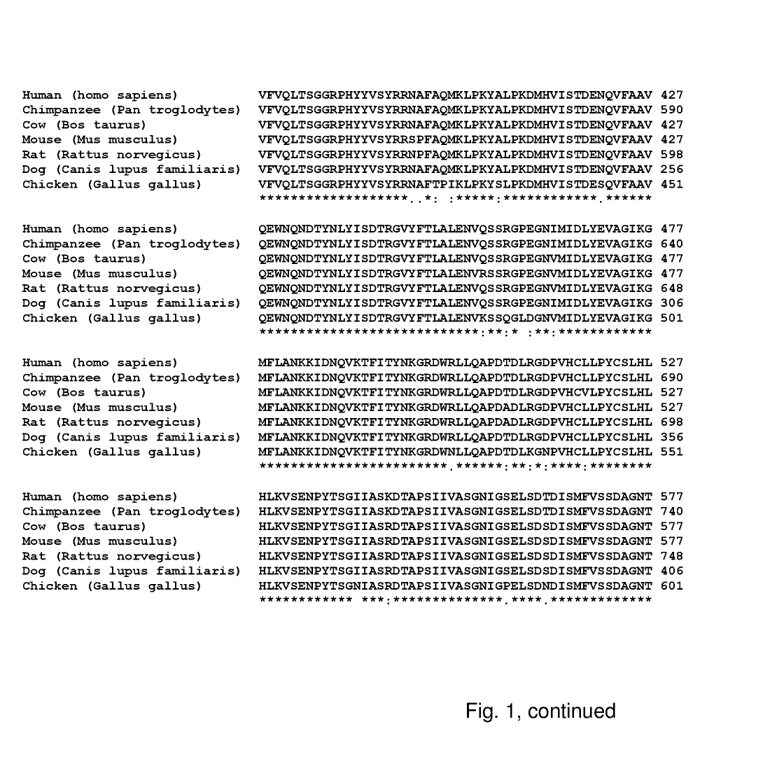

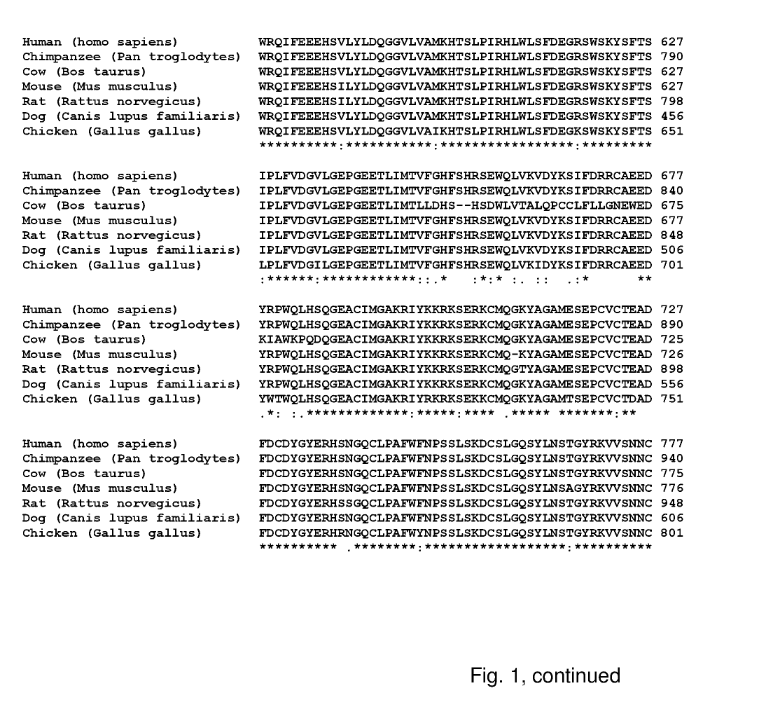

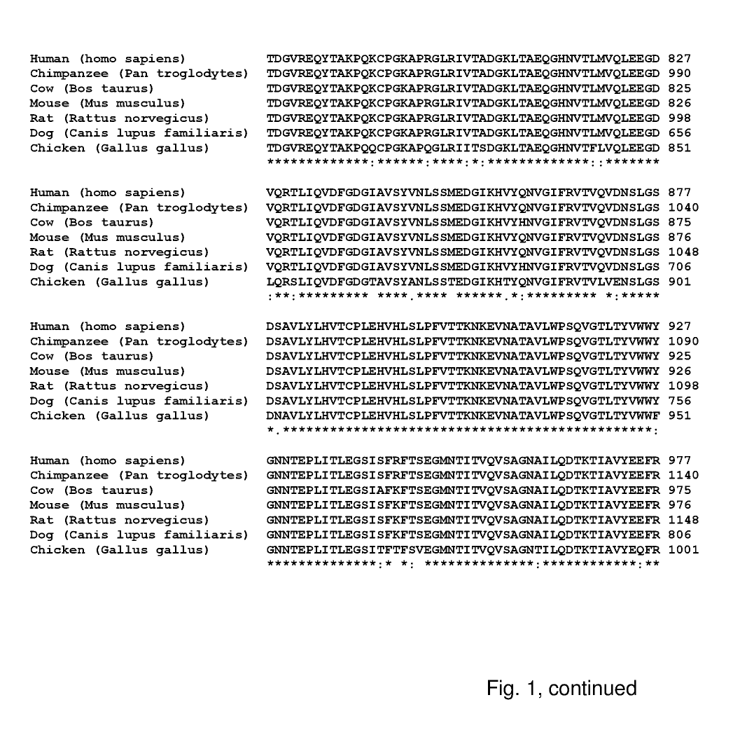

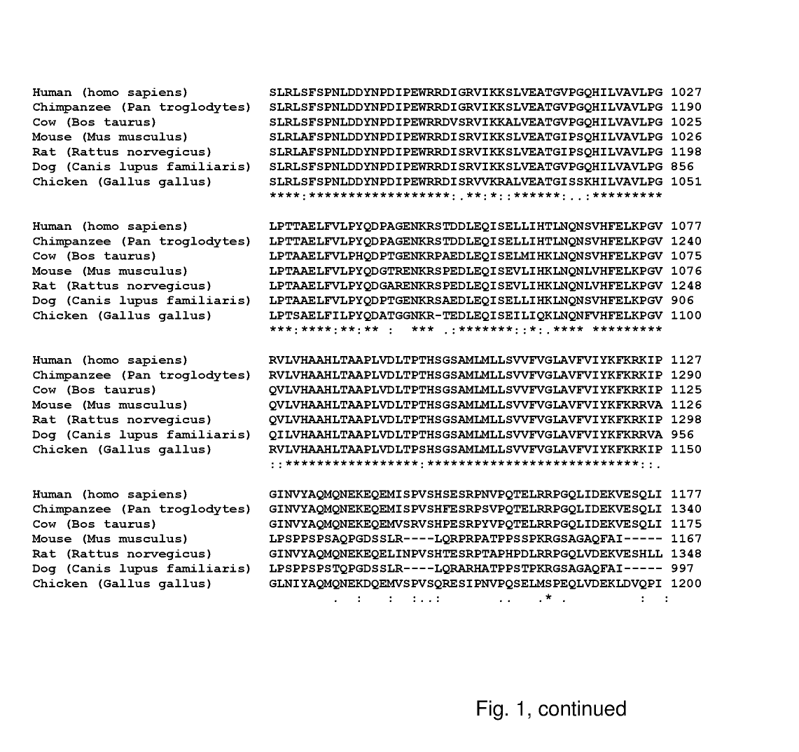

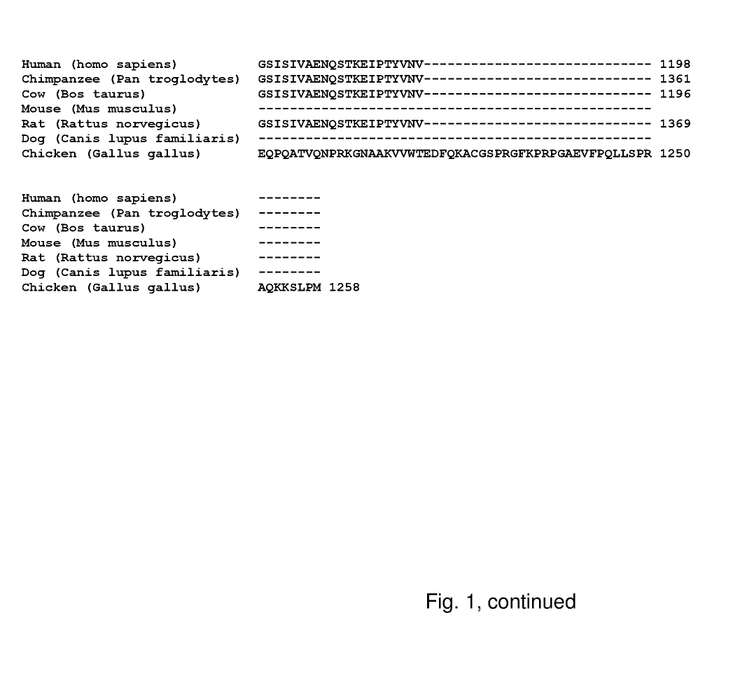

Using gene array analysis of adipose tissue from SorCS1 knockout wild-type adipose mice the inventors tested expression of A) 84 genes related to the mouse insulin signalling pathway and B) 84 genes related to mouse lipoprotein signalling & cholesterol metabolism. In practice, first strand cDNA was synthesized from total RNA (Applied Biosystems) from SorCS1 knockout (−/−) and wild-type (+/+) adipose tissue from female mice 50 weeks of age (n=3). Then superarray of A) Mouse Insulin Signalling Pathway (PAMM-030A RT2 Profiler PCR arrays) or B) the type Mouse Lipoprotein Signalling & Cholesterol Metabolism (PAMM-080-A RT2 Profiler PCR arrays) were processed using an ABI7900 platform (Applied Biosystems) and SYBR Green/Rox PCR (SABiosciences). AROS Applied Biotechnology, Aarhus, Denmark, did the expression analyses. Genes showing an expression more than 3 times up- or down-regulated in the SorCS1 knockout mice when compared to wild-type mice are listed in the upper tables and their known functions are indicated in the table below. Several genes in A and B show changed expression in the SorCS1 knockout mice compared to the wild-type mice indicating that insulin and cholesterol signalling pathways and metabolism are altered in SorCS1 knockout mice.

FIG. 3: Reduced weight in diabetic db/db mice after over-expression of soluble SorCS1.

To evaluate the effect of soluble SorCS1 on weight in an obese mouse model that spontaneously develops type 2 diabetes, we used the db/db mouse strain (BKS.Cg-m+/+Lpr^{db}/BomTac from Taconic). These mice lack the leptin receptor and consequently the mice become obese and develop insulin resistance and finally severe diabetes at the age of 6-8 weeks. The inventors injected adenovirus expressing either human soluble (hsol.) SorCS1 or LacZ as a control (as described in example 2), to examine the effect on weight. In detail, db/db female mice 6 weeks of age were injected in the tail vein with 2E9 pfu's of an adenoviral vector with either hsol.SorCS1 or LacZ (from ViraQuest Inc, North Liberty, Iowa) as a negative control virus. In the morning, on day 0, 9, 14 and day 16, the mice were weighed on a scale. Data are means±SEM for 5 mice in each group. On day 9 to 16, the db/db female mice with over-expression of soluble SorCS1 exhibited a significant decrease in weight compared to the mice that received the control LacZ virus. Thus, over-expression of soluble SorCS1 improves the obese status in this obese mouse model.

FIG. 4: Reduced food intake and weight in diabetic db/db mice after over-expression of soluble SorCS1.

To evaluate the effect of soluble SorCS1 on body weight in an obese mouse model that spontaneously develops type 2 diabetes, the inventors used the db/db mouse strain (BKS.Cg-m+/+Lpr^{db}/BomTac from Taconic). These mice lack the leptin receptor and consequently the mice become obese and develop insulin resistance and finally severe diabetes at the age of 6-8 weeks. The inventors injected adenovirus expressing either hsol.SorCS1 or LacZ as a control (as described in example 2), to examine the effect on weight. In detail, db/db female mice 6 weeks of age were injected in the tail vein with 2E9 pfu's of an adenoviral vector with either hsol.SorCS1 or LacZ (from ViraQuest Inc, North Liberty, Iowa) as a negative control virus. A) In the morning of day 9 after virus treatment each mouse was moved to a metabolic cage with a measured amount of food. 24 hours later the mouse was moved back to a normal mouse cage and the food in the metabolic cage was weighed to determine the food intake. The amount of ingested food over 24 hours is shown. Data are means±SEM for 4 mice in each group. Mice with over-expression of soluble SorCS1 ate significant less than the control mice expressing LacZ. B) In the morning, on day 0 and 11 after virus treatment, the mice were weighed on a scale. The relative weight changes over the time period are shown. Data are means±SEM for 4 mice in each group. On day 11, the db/db female mice with over-expression of soluble SorCS1 exhibited a significant decrease in body weight compared to the mice that received the control LacZ virus.

FIG. 5: Reduced food intake and weight in obese DIO male mice after over-expression of soluble SorCS1.

Obese and pre-diabetic “diet induced obesity” (D10) male mice 15 weeks of age were injected i.v with adenovirus encoding the soluble extra-cellular domain of SorCS1 or LacZ (control). A) At day 10 each group of virus treated mice were placed in cages and the food intake over the next 4 days was measured every 24 hrs. Mice with over-expression of soluble SorCS1 ingested significantly less than the control mice expressing LacZ, both 11 and 14 days after virus injection. B) At day 0, 11 and 14 after virus treatment the mice were weighed. The relative weight changes over the time period are shown. In conclusion over-expression of soluble SorCS1 leads to a significant weight reduction compared to the lacZ control (P<0.05 SorCS1 vs LacZ).

FIG. 6: Reduced food intake and weight in obese and diabetic ob/ob female mice after over-expression of soluble SorCS1.

Obese ob/ob mice 8 weeks of age, with spontaneous type-2 diabetes, were injected i.v with adenovirus encoding the soluble extra-cellular domain of SorCS1 or LacZ (control). A) At day 9 the mice were placed in metabolic cages and the food intake over the next 24 hrs was measured. Mice with over-expression of soluble SorCS1 ate significant less than the control mice expressing LacZ. B) At day 0 and 10 after virus treatment the mice are weight. The relative weight changes over the time period are shown. In conclusion over-expression of soluble SorCS1 leads to a significant weight reduction.

FIG. 7. Overexpression of human soluble SorCS1 by adenovirus increase expression of PRDM16 and PGC-1alpha in adipose tissue from db/db mice. Db/db mice were i.v. injected with 2E9 PFU/mouse of either AV-hsol.sorcs1 or AV-LacZ and gonadal fat was harvested 14 days post injection. After isolation of mRNA from the gonadal fat, a qPCR of specific fat genes was performed for CD137 (brite adipose tissue marker), PRDM16 and PGC-1α (brown adipose tissue markers), and GAPDH as a household gene. Several proteins are involved in the process of converting WAT to BAT in mice, e.g. PRDM16 and PGC-1 alpha. PRDM16 is selectively expressed in BAT, where it activates BAT-specific gene expression and represses WAT-specific gene expression, through an interaction with the co-receptor PGC-1alpha. mRNA from PRDM16 and PGC-1alpha are more than 2-fold upregulated in the adipose tissue from db/db mice subjected to AV-hsol.sorcs1 virus, p<0.05 (student's t-test, 2 tailed, 2 sample, equal variance).

FIG. 8. Less weight gain in animals, on normal chow (ND), treated with human soluble SorCS1 expressed by adenoassociated virus. Mice (C57BL6/j bom tac) (n=5-6 per group) were i.v. injected with either soluble human SorCS1 (AAV-hsol.sorcs1) or LacZ (AAV-LacZ) adenoassociated virus (AAV). The titer of virus injected was 1 E11 vgc/mouse (vgc=viral genome copies). The mice were weighed every fortnight. Mice treated with AAV-hsol.sorcs1 gained 32% less weight on normal chow in 150 days, as compared to the LacZ control group. The effect of the virus on weight gain lasts up to 150 days post injection of virus (p=0.0296, 2-way ANOVA, treatment).

### AGENT OF THE INVENTION AND MEDICAL USES THEREOF

The present invention in various aspects concerns the Vps10p-domain receptors SorCS1 and SorCS3 such as polypeptides comprising the amino acids selected from the group SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64

In one aspect the invention concerns a polypeptide having an amino acid sequence selected from the group consisting of SEQ ID NO: 61, 62, 63 and 64. In one embodiment the polypeptide has the amino acid sequence of SEQ ID NO: 61. In another embodiment the polypeptide has the amino acid sequence of SEQ ID NO: 62. In yet another embodiment the polypeptide has the amino acid sequence of SEQ ID NO: 63. In yet another embodiment the polypeptide has the amino acid sequence of SEQ ID NO: 64. In one embodiment the invention concerns any one of the polypeptides selected from the group consisting of SEQ ID NO: 61, 62, 63 and 64 for medical use.

The present inventors have found that overexpression of soluble SorCS1 in a subject results in decreased body weight of the subject. The inventors have also found that overexpression of soluble SorCS1 in mice decreases the desire of the mice to eat, i.e. reduces appetite.

The present inventors have studied the effect of administration of soluble SorCS1 in mice. The inventors have surprisingly found that following SorCS1 administration the mice loose weight as compared to control. Without being bound by theory, the weight loss has been correlated to a reduced desire to eat in the subjects having received SorCS1 treatment. Additionally the inventors have found that mice treated with SorCS1 exhibits a higher rate of metabolism, and has a higher degree of brown fat as compared to control mice receiving LacZ. Brown fat has a higher degree of mitochondria than white fat, and thus brown adipose tissue produces more heat than white adipose tissue. Consequently the present invention in one aspect also concern use of the SorCS1 agent of the present invention for increasing thermogenesis.

Specifically the inventors have demonstrated that a sixteen days treatment with hepatic adenoviral as well as adeno-associated viral overexpression of soluble SorCS1 (extracellular domain; prepro-soluble-SorCS1; SEQ ID NO: 15) results in a weight reduction of about 23% compared to mice treated with a control virus. The weight reduction is at least partly due to appetite suppression as food intake in the same period also was reduced compared to control mice. The weight reduction may also be related to an increased overall metabolism following SorCS1 treatment. Prepro-soluble-SorCS1 (SEQ ID NO: 5) is converted into active mature soluble SorCS1 (SEQ ID NO: 15) following administration by vivo posttranslational modification.

Consequently in a main aspect the present invention relates to an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c), for use in a method for reducing appetite, and/or for promoting weight loss, and/or treating obesity, and/or increasing metabolism, and/or increasing thermogenesis, and/or converting white fat into brown fat.

In another aspect the present invention relates to an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 150 contiguous amino acids of any of i) and ii) wherein any amino acid specified in the chosen sequence is changed to a different amino acid, provided that no more than 30 of the amino acid residues in the sequence are so changed,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for reducing appetite, and/or for promoting weight loss, and/or treating obesity, and/or increasing metabolism, and/or increasing thermogenesis, and/or converting white fat into brown fat.

In one aspect the invention concern the use of an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
for the preparation of a medicament for reduction of appetite, and/or for promoting weight loss, and/or increasing metabolism, and/or increasing thermogenesis, and/or converting white fat into brown fat and/or for treating obesity.

In one aspect the invention concerns a method for reducing appetite, and/or for promoting weight loss, and/or for treating obesity, and/or for increasing metabolism, and/or for increasing thermogenesis, and/or for converting white fat into brown fat, the method comprising administering to an individual in need thereof a therapeutically effective amount of an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In one aspect the invention concerns a method for treating obesity the method comprising administering to an individual in need thereof a therapeutically effective amount of an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In one aspect the invention concerns a method for increasing metabolism, the method comprising administering to an individual in need thereof a therapeutically effective amount of an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In one aspect the invention concerns a method for increasing thermogenesis in a mammal, the method comprising administering to the mammal a therapeutically effective amount of an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In one aspect the invention concerns an in vivo method for converting white fat into brown fat, the method comprising administering to a mammal a therapeutically effective amount of an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In another aspect the invention concerns an in vitro method for converting white fat into brown fat, the method comprising contacting a cell with an effective amount of an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In another aspect the present invention concerns an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for the cosmetic treatment of obesity.

In one embodiment the agent of the invention is for cosmetic use in general, e.g. by reduction of local fat by local application to a mammal such as a human being, of a formulation comprising the agent of the present invention.

In another aspect the invention concerns a method for supporting weight loss comprising administering a functional food or dietary supplement comprising the agent of the present invention.

In conjunction with the present studies the inventors found that even high overexpression of SorCS1 does not cause hypoglycemia in euglycemic mice. Accordingly SorCS1 can be used to treat overweight and/or obese patients which patients are not afflicted with insulin resistance or diabetes.

Thus in one embodiment the agent of the present invention is for use in non-diabetic patients, i.e. patients who are not suffering from any type of diabetes, e.g. patients who are not suffering from type II diabetes.

In another embodiment the agent of the present invention is for use in non-insulin resistant patients, i.e. patients which are not afflicted with insulin resistance.

In one embodiment the subject receiving therapy with the agent of the present invention does not suffer from insulin resistance and/or diabetes mellitus type 2.

In one embodiment the agent of the present invention is for use in a combination treatment of obesity and insulin resistance.

In another embodiment the agent of the present invention is for use in a combination treatment of obesity and type II diabetes.

In another embodiment the agent of the present invention is for use in a combination treatment of over-weight and insulin resistance.

In certain embodiments it may be relevant with a combination treatment either to obtain enhanced effect of the condition to be treated or to effectively target multiple conditions as defined above. Thus the agent according to the invention may be administered with at least one other compound.

In one embodiment the agent of the present invention is a polypeptide variant, wherein any amino acid specified in the selected sequence is altered to provide a conservative substitution.

In one embodiment of the present invention the agent as defined herein is a polypeptide having at least 65%, more preferably at least 70%, more preferably at least 75%, preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% sequence identity to an amino acid sequence selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64.

The agent of the invention is preferably a human SorCS1 polypeptide either in mature form or having an intact signal peptide (pre-domain) and/or pro-domain peptide. In one embodiment the agent is a polypeptide selected from the group consisting of SEQ ID NOs: 1, 2, 3, 4, 6, 7, 8, 9, 11, 12, 13 and 14.

In another embodiment the agent is a non-human polypeptide selected from the group consisting of SEQ ID NOs: 16, 17, 18, 19, 20, 22, 26, 28, 29, 30, 31 and 32.

In one embodiment, the active polypeptide of the present invention as defined above is selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64.

Biologically active variants of the above listed amino acid sequences are also considered to fall within the scope of the present invention. Accordingly in one embodiment the polypeptide is a variant polypeptide, wherein any amino acid specified in the selected sequence is altered to provide a conservative substitution as defined above. Accordingly, the polypeptide preferably has at least 40%, such as at least 41%, such as at least 42%, such as at least 43%, such as at least 44%, such as at least 45%, such as at least 46%, such as at least 47%, such as at least 48%, such as at least 49%, e.g. 50%, such as at least 51%, such as at least 52%, such as at least 53%, such as at least 54%, such as 55%, such as at least 56%, such as at least 57%, such as at least 58%, such as at least 59%, e.g. 60%, such as 61%, e.g. 62%, such as 63%, e.g. 64%, such as 65%, such as at least 66%, such as at least 67%, such as at least 68%, such as at least 69%, e.g. 70%, such as at least 71%, such as at least 72%, such as at least 73%, such as at least 74%, e.g. 75%, such as at least 76%, such as at least 77%, such as at least 78%, such as at least 79%, such as 80%, such as at least 81%, such as at least 82%, such as at least 83%, such as at least 84%, e.g. 85%, such as at least 86%, such as at least 87%, such as at least 88%, such as at least 89%, such as 90%, such as at least 91%, such as at least 92%, such as at least 93%, such as at least 94%, e.g. 95%, such as such as at least 96%, such as at least 97%, such as at least 98%, e.g. at least 99% such as 100% sequence identity to a protein having a sequence selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64.

In one embodiment, the polypeptide is a naturally occurring allelic variant of the sequence selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64 and preferably the polypeptide comprises an amino acid sequence selected from the group consisting of: SEQ ID NOs: 15, 5, 10, 21, 27, 33, 37, 39, 43 and 47.

More preferably the agent of the invention is a polypeptide variant having at least 40%, such as at least 45%, e.g. 50%, such as 55%, e.g. 60%, such as 65%, e.g. 70%, e.g. 75%, such as 80%, e.g. 85%, such as 90%, e.g. 95%, such as 98%, e.g. 99% sequence identity to a protein having a sequence selected from the group consisting of SEQ ID NOs: 15, 5, 64, 62, 10, 21, 27, 33, 37, 39, 43 and 47

Polypeptides expressed in eukaryotic cells are often glycosylated, such as N- or O-glycosylated. The glycosylation pattern is important for interaction of the folded polypeptide with other molecules and affects the polarity of the polypeptide.

Thus in one embodiment the polypeptide agent of the invention is glycosylated, such wherein the agent is a polypeptide selected from the group consisting of SEQ ID NOs: 1, 2, 3, 4, 6, 7, 8, 9, 11, 12, 13, 14, wherein the polypeptide may be glycosylated in one or more of the following amino acid residue positions 184, 352, 433, 765, 776, 816, 847, 908 and 929, and/or wherein the polypeptide is selected from the group consisting of SEQ ID NOs: 16, 17, 18, 19, 20, 22, 26, 28, 29, 30, 31 and 32, wherein the polypeptide may be glycosylated in one or more of the following amino acid residue positions 184, 352, 433, 765, 776, 816, 847, 908 and 929, and in another embodiment the glycosylated fragment has the sequence selected from the group consisting of SEQ ID NO: 5, 10 and 15, or the glycosylated polypeptide fragment has the sequence selected from the group consisting of SEQ ID NO: 21, 27 and 33.

In one embodiment the polypeptide is N-glycosylated in one or more asparagin amino acid residues corresponding to amino acid positions 184, 352, 433, 765, 776, 816, 847, 908 and 929 of SEQ ID NO: 1 or equivalent positions in post-translationally modified variants of SEQ ID NO: 1.

In some embodiments, however, it is preferred that the polypeptide expressed is subsequently deglycosylated. This may be achieved by methods known by the person of skill in the art.

While native SorCS1 and the other native Vps10p-domain receptors are Type-I membrane proteins it is preferred that the agent of the invention has been genetically modified, such as C-terminally truncated to remove the single transmembrane helix and the intracellular C-terminal. Thus in one embodiment the agent of the invention comprises a soluble fragment of a polypeptide as defined herein or a fragment of a variant, and accordingly. In one such embodiment the polypeptide is a soluble polypeptide being a fragment of the sequences selected from the group consisting of SEQ ID NOs: 1, 2, 3, 4, 6, 7, 8, 9, 11, 12, 13, 14, or the polypeptide is a soluble polypeptide being a fragment of the sequences of SEQ ID NO: 15.

In certain embodiments it may be advantageous to enhance the intramolecular stability by forming cystein bridges. In one embodiment the polypeptide as defined herein is capable of forming at least one intramolecular cystin bridge. Occasionally it is advantageous for stability and efficacy to administer a multimer such as a dimer of the polypeptides of the invention. In one embodiment the polypeptide as defined herein above comprises a dimer of said polypeptide linked through at least one intermolecular cystin bridge.

The polypeptide of the invention may comprise a tag useful for purification. In one embodiment the polypeptide according to the present invention comprises an affinity tag, such as a polyhis tag, a GST tag, a HA tag, a Flag tag, a C-myc tag, a HSV tag, a V5 tag, a maltose binding protein tag, a cellulose binding domain tag. In addition to affinity tags, the polypeptide of the invention may also comprise tags altering the functionallity of the polypeptide such as tags or conjugated groups altering the plasma and/or serum half-life of SorCS1 administered to a mammal as discussed herein below in the section concerning agents of the invention having increased half-life.

Medical Use of Other Vps10p-Domain Receptors

As indicated above, the invention is not limited to mature soluble SorCS1, but can be any biologically active sequence variant thereof as well as nucleotides encoding SorCS1 or a fragment or variant thereof, including vectors comprising the nucleotide encoding the SorCS1 polypeptide. Thus in one embodiment the invention relates to a nucleic acid sequence encoding a polypeptide as defined above for use in the supression of appetite, reduction of hunger and/or reduction of prospective consumption and/or reduction of the desire to eat, and/or increasing satiety, and/or treatment of obesity, and/or for promoting weight loss, and/or increasing metabolism, and/or increasing thermogenesis, and/or converting white fat into brown fat. The invention also concerns cells comprising the nucleic acid sequence or the above expression vector.

Variants of SorCS1 as defined in the present invention may in certain embodiments include full length or fragments of other Vps10p-domain receptors.

TABLE 3
Sequence identity between human full length SorCS1
and other full length Vps10p-D receptors

| Name | % identity | SEQ ID NO: |
|---|---|---|
| SorCS1 | 100 | 5 |
| SorCS2 | 43 | 54 |
| SorCS3 | 64 | 55 |
| Sortilin | 18 | 52 |
| SorLA | 13 | 53 |

In one embodiment the agent of the present invention is SorCS3. Accordingly, in one aspect the present invention relates to an agent selected from the group consisting of:
a) an isolated polypeptide comprising:
   i) the amino acid sequence of SEQ ID NOs: 55, 56, 57, 58, 59 and 60; or
   ii) a biologically active sequence variant of the amino acid sequence of i) wherein the variant has at least 60% sequence identity to said SEQ ID NO: 55, 56, 57, 58, 59 and 60,
   iii) a biologically active fragment of at least 15 contiguous amino acids of any one of i) through ii), said fragment having at least 60% sequence identity to SEQ ID NO: 55, 56, 57, 58, 59 and 60 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for reducing appetite, and/or for promoting weight loss, and/or treating obesity, and/or increasing metabolism, and/or increasing thermogenesis, and/or converting white fat into brown fat.

In one embodiment the agent is a biologically active fragment of any one of SEQ ID NO: 55, 56, 57, 58, 59 and 60, wherein the fragment comprises less than 500 contiguous amino acid residues, such as less than 450 contiguous amino acid residues, for example less than 400 contiguous amino acid residues, such as less than 350 contiguous amino acid residues, for example less than 300 contiguous amino acid residues, for example less than 250 contiguous amino acid residues, such as less than 240 contiguous amino acid residues, for example less than 225 contiguous amino acid residues, such as less than 200 contiguous amino acid residues, for example less than 180 contiguous amino acid residues, such as less than 160 contiguous amino acid residues, for example less than 150 contiguous amino acid residues, such as less than 140 contiguous amino acid residues, for example less than 130 contiguous amino acid residues, such as less than 120 contiguous amino acid residues, for example less than 110 contiguous amino acid residues, such as less than 100 contiguous amino acid residues, for example less than 90 contiguous amino acid residues, such as less than 85 contiguous amino acid residues, for example less than 80 contiguous amino acid residues, such as less than 75 contiguous amino acid residues, for example less than 70 contiguous amino acid residues, such as less than 65 contiguous amino acid residues, for example less than 60 contiguous amino acid residues, such as less than 55 contiguous amino acid residues, for example less than 50 contiguous amino acid residues, such as less than 45 contiguous amino acid residues, for example less than 40 contiguous amino acid residues, such as 35 contiguous amino acid residues, for example 30 contiguous amino acid residues, such as 25 contiguous amino acid residues, such as 20 contiguous amino acid residues, for example 15 contiguous amino acid residues of an any one of the amino acid sequences selected from the group consisting of SEQ ID NOs: SEQ ID NO: 55, 56, 57, 58, 59 and 60.

In another embodiment the agent is a biologically active fragment of any one of SEQ ID NO: 55, 56, 57, 58, 59 and 60, wherein the fragment comprises at least 15 contiguous amino acid residues, such as more than 20 contiguous amino acid residues, for example more than 25 contiguous amino acid residues, for example more than 50 contiguous amino acid residues, such as more than 75 contiguous amino acid residues, for example more than 100 contiguous amino acid residues, such as more than 125 contiguous amino acid residues, for example more than 150 contiguous amino acid residues, such as more than 175 contiguous amino acid residues, for example more than 200 contiguous amino acid residues, such as more than 225 contiguous amino acid residues, for example more than 250 contiguous amino acid residues, such as more than 275 contiguous amino acid residues, for example more than 300 contiguous amino acid residues, such as more than 325 contiguous amino acid residues, for example more than 350 contiguous amino acid residues, such as more than 375 contiguous amino acid residues, for example more than 400 contiguous amino acid residues, such as more than 425 contiguous amino acid residues, for example more than 450 contiguous amino acid residues, such as more than 475 contiguous amino acid residues, for example more than 500 contiguous amino acid residues, such as more than 525 contiguous amino acid residues, for example more than 550 contiguous amino acid residues, such as more than 575 contiguous amino acid residues, for example more than 600 contiguous amino acid residues, such as more than 625 contiguous amino acid residues, for example more than 650 contiguous amino acid residues, such as more than 675 contiguous amino acid residues, such as more than 700 contiguous amino acid residues of any one of the amino acid sequences selected from the group consisting of SEQ ID NOs: SEQ ID NO: 55, 56, 57, 58, 59 and 60.

In one embodiment the agent of the present invention is SorCS2. Accordingly, in one aspect the present invention relates to an agent selected from the group consisting of:
a) an isolated polypeptide comprising:
   i) the amino acid sequence of SEQ ID NOs: 54; or
   ii) a biologically active sequence variant of the amino acid sequence of i) wherein the variant has at least 60% sequence identity to said SEQ ID NO: 54,
   iii) a biologically active fragment of at least 15 contiguous amino acids of any one of i) through ii), said fragment having at least 60% sequence identity to SEQ ID NO: 54 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for reducing appetite, and/or for promoting weight loss, and/or treating obesity, and/or increasing metabolism, and/or increasing thermogenesis, and/or converting white fat into brown fat.

In one embodiment the agent of the present invention is Sortilin. Accordingly, in one aspect the present invention relates to an agent selected from the group consisting of:
a) an isolated polypeptide comprising:
   i) the amino acid sequence of SEQ ID NOs: 52; or
   ii) a biologically active sequence variant of the amino acid sequence of i) wherein the variant has at least 60% sequence identity to said SEQ ID NO: 52,
   iii) a biologically active fragment of at least 15 contiguous amino acids of any one of i) through ii), said fragment having at least 60% sequence identity to SEQ ID NO: 52 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for reducing appetite, and/or for promoting weight loss, and/or treating obesity, and/or increasing metabolism, and/or increasing thermogenesis, and/or converting white fat into brown fat.

### Obesity Associated Disorders

In certain aspects the present invention concern obesity associated disorders such as obesity associated sleep disorders, e.g. obesity related breathing disorders.

Accordingly, in one embodiment the invention concerns an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for treating or preventing or reducing incidence of a sleep-related breathing disorder in an individual in need thereof.

In one embodiment the obesity-associated and/or sleep-related breathing disorder is selected from central sleep apnea (CSA), Cheyne-Stokes breathing-central sleep apnea (CSB-CSA), obesity hypoventilation syndrome (OHS), congenital central hypoventilation syndrome (CCHS), obstructive sleep apnea (OSA) and idiopathic central sleep apnea (ICSA).

In one aspect the present invention concerns an agent selected from the group consisting of:
a) an isolated polypeptide comprising:
   i) the amino acid sequence of SEQ ID NOs: 15; or
   ii) a biologically active sequence variant of the amino acid sequence of i) wherein the variant has at least 60% sequence identity to said SEQ ID NO: 15,
   iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for use in a method for treating or preventing or reducing incidence of an obesity associated disorder selected from the group consisting of non-alcoholic fatty liver disease, sleep apnea, obesity associated metabolic disorders e.g. osteoarthritis, unwanted weight gain or body mass index and excessive appetite resulting in unwanted weight gain.

### Agents of the Invention Having Increased Half-Life

One approach to improve the efficacy of a therapeutic protein such as SorCS1 or SorCS3 of the present invention is to increase its serum persistence, thereby allowing higher circulating levels, and/or allowing circulating levels to be present for a longer time thereby providing higher exposure (AUC), less frequent administration and reduced doses.

In determining bioequivalence, for example, between two products such as a commercially-available product and a candidate drug, pharmacokinetic studies are conducted whereby each of the preparations are administered in a cross-over study to volunteer subjects, generally healthy individuals but occasionally in patients. Serum/plasma samples are obtained at regular intervals and assayed for parent drug (or occasionally metabolite) concentration. Occasionally, blood concentration levels are neither feasible nor possible to compare the two products, then pharmacodynamic endpoints rather than pharmacokinetic endpoints are used for comparison. For a pharmacokinetic comparison, the plasma concentration data are used to assess key pharmacokinetic parameters such as area under the curve (AUC), peak concentration (Cₘₐₓ), time to peak concentration (Tₘₐₓ), and absorption lag time (t_{lag}). Testing can be conducted at several different doses, especially when the drug displays non-linear pharmacokinetics.

In addition to data from bioequivalence studies, other data may need to be submitted to meet regulatory requirements for bioequivalence. Such evidence may include analytical method validation and/or in vitro-in vivo correlation studies (IVIVC).

In one particular embodiment, the agent of the invention, such as the polypeptide of the invention is modified in order to provide higher exposure (AUC), less frequent administration and reduced doses.

In another embodiment, the agent of the invention, such as the polypeptide of the invention is modified in order to increase its half-life when administered to a patient, in particular its plasma half-life. In particular, the agent, such as the polypeptide is modified in order to increase its plasma halflife. A number of methods are available in the art for modification of peptide drugs in order to increase its halflife, and such methods of the art can be employed for modification of the SorCS1 polypeptides of the present invention and variants thereof. Short plasma half-life times are often caused by fast renal clearance as well as enzymatic degradation occurring during systemic circulation. Modifications of the peptide/protein can lead to prolonged plasma half-life times. Increased halflife can for example be obtained by shortening the overall amino acid amount of the polypeptide.

Exopeptidases is a prominent group of proteolytic enzymes occurring in plasma, liver and kidney, which affect therapeutic peptides and proteins. Thus, modification of either or both of the peptide drug termini in many cases increase enzymatic stability, and thus plasma halflife. Thus, in one approach, one or more additional compounds are coupled to a polypeptide of the present invention, in order to increase its plasma halflife. In one embodiment, the terminal modification is N-acetylation and/or C-amidation. In another such embodiment, The N and/or C-terminus is conjugated to polyethylenglycol (PEG) compounds. One specific modification of the polypeptide is the dual modification of N-terminal palmitoyl and C-terminal PEGylation. A headto-tail cyclization of the polypeptide drug by the formation of an amide bond between C- and N-terminus is also possible in order to prevent exopeptidase caused degradation of the SorCS1 polypeptide.

In another embodiment, increased plasma halflife is obtained by replacement of one or more amino acids, which are known to be susceptible to enzymatic cleavage, thereby letting the polypeptide escape proteolytic degradation. For example, one or more L-amino acids could be substituted with D-amino acids at one or both polypeptide termini, and/or within the polypeptide in order to avoid degradation, and thereby increase plasms halflife.

Increased halflife of the polypeptide of the invention can also be obtained by coadministration of the polypeptide with one or more specific enzyme inhibitors. Such enzyme inhibitors could be included in the kit-of-parts of the invention. In yet another approach, increased halflife could be obtained by increasing the molecular mass of the SorCS1 polypeptide of the invention.

As a general rule, substances with a molecular mass below 5 kDa which are not bound to plasma proteins are excreted via the renal route, whereas molecules with a molecular mass over 50 kDa cannot or only in very small amounts be found in the glomerular ultrafiltrate. Accordingly, a main reason for short peptide and protein half-life time beside enzymatic degradation is their fast renal excretion. Therefore, half-life time can be prolonged by increasing the polypeptide drug size. Furthermore, a synergistic effect may be given by additional enzyme inhibition. Beside chemical modification of N- and C-termini which is an effective way to inhibit exopeptidases and replacement of labile amino acids, PEGylation allows to specifically protect endangered termini and furthermore increases molecular mass. In addition, PEGylation within the drug molecule expectedly leads to improved enzymatic stability mediated by a steric hindrance of proteolytic enzymes.

Poly(ethyleneglycol) (PEG) exhibits several beneficial properties: high water solubility, high mobility in solution, lack of toxicity and immunogenicity and ready clearance from the body. Very often these properties are transferred to PEG-protein or PEG-peptide conjugates. The extent of these feature are dependent on the molecular weight of the attached PEG.

Also polymers of N-acetylneuraminic acid (polysialic acids) may be used as conjugates to a polypeptide of the invention. Polysialic acids are naturally occurring, biodegradable, highly hydrophilic compounds which have no known receptors in the human body. PEGylation and sialyation prolong half-life time by a combination of two mechanisms—improvement of enzymatic stability and decrease of renal excretion by increasing molecular mass.

Albumin is known to have a long plasma half-life and because of this property it has been used in drug delivery in order to increase halflife of drugs. For this purpose albumin has been conjugated to such pharmaceutical compounds. Especially suitable is coupling to the free cysteine residue on the albumin molecule (Cys 34), e.g. by methods described in WO2010092135, especially the methods using PDPH (3-(2-pyridyldithio) propionyl hydrazide) to link albumin to a SorCS polypeptide of the invention including fragments thereof via a hydrazone link to the SorCS1 polypeptide. Another coupling technology is described by Neose (see eg US2004/0126838) using enzymatic glycoconjugation. This technology can be used to link e.g. albumin to a SorCS1 polypetide of the invention using a suitable linker.

In certain embodiments the present invention concerns a long-acting modified SorCS1 polypeptide wherein said modified polypeptide comprises a mammalian SorCS1 or analog thereof linked to a pharmaceutically acceptable molecule, e.g. human SorCS1 linked to, e.g. fused to, albumin, or fused to a fatty acid of suitable length, or fused to an Fc fragment of a mammalian antibody, or a variant of an Fc fragment of a mammalian antibody or conjugated to an acylation group or PEG, that in some embodiments provides an in vivo plasma half-life of the mammalian SorCS1 or analog thereof, or the modified SorCS1 which is from 2 to 48 hours or longer, typically from 4 to 28 hours, such as 6-8 hours in a mammal.

The creation of fusion proteins comprised of immunoglobulin constant regions linked to a protein of interest, or fragment thereof, has been described (see, e.g., U.S. Pat. Nos. 5,155,027, 5,428,130, 5,480,981, and 5,808,029). These molecules usually possess both the biological activity associated with the linked molecule of interest as well as the effector function, or some other desired characteristic, associated with the immunoglobulin constant region. Fusion proteins comprising an Fc portion of an immunoglobulin can bestow several desirable properties on a fusion protein including increased stability, increased serum half-life (see Capon et al. (1989) Nature 337:525) as well as binding to Fc receptors such as the neonatal Fc receptor (FcRn) (U.S. Pat. Nos. 6,086,875, 6,030,613, and 6,485,726).

In one embodiment the moiety resulting in increased half-life is a multifunctional moiety, such as bi- or trifunctional, which may be covalently linked to one or more SorCS1 molecules, such as one or more mammalian SorCS1 molecule, and covalently linked to one or more pharmaceutically acceptable molecule(s) so as to create the modified SorCS1 compound. The linker may be stabile which means that no significant chemical reactions, e.g. hydrolysis, occurs at physiological conditions (e.g. temperature of 37° C. and pH 7.4) over the time period of the treatment. This can be determined by stability studies known in the art. The linker may be a chemical linker meaning that it is generated by organic chemistry outside a living cell. The linker may be a sugar moiety, such as a glycosylation on a protein, or may be chemically prepared and used to link the SorCS1 molecule, and a second pharmaceutically acceptable molecule such as PEG variants, albumin, fatty acids or antibodies or antibody fragments such as Fc fragments.

In one embodiment, the agent, such as SorCS1 polypetide, of the invention is coupled to a immunoglobulin-Fc such as IgG-Fe.

The SorCS1 compound of the present invention may optionally comprise at least one peptide linker. In one embodiment, the linker is comprised of amino acids linked together by peptide bonds, wherein the amino acids are selected from the twenty naturally occurring amino acids. In various embodiments the linker can comprise 1-5 amino acids, 1-10 amino acids, 1-20 amino acids, 10-50 amino acids, 50-100 amino acids, or 100-200 amino acids. In one embodiment the amino acids are selected from glycine, alanine, proline, asparagine, glutamine, and lysine. In one embodiment a linker is made up of a majority of amino acids that are sterically unhindered, such as glycine and alanine. The linker in one embodiment can comprise the sequence Gn (equivalently, -(Gly)n-). The linker can in one embodiment comprise the sequence (GGS)n or (GGGGS)n. In each instance, n is an integer, such as 1, 2, 3, 4, 5, 6, 7, 8, 9, or 10. Examples of linkers include, but are not limited to, GGG, SGGSGGS (SEQ ID NO:65), GGSGGSGGSGGSGGG (SEQ ID NO:66),GGSGGSGGSGGSGGSGGS (SEQ ID NO:67), GGGGSGGGGSGGGGS (SEQ ID NO:68) and EFAGAAAV (SEQ ID NO:69).

In one embodiment the peptide linker has at least 1 amino acid, such as from 1-200 amino acids, typically 1-50 amino acids wherein the amino acids are selected from the twenty naturally occurring amino acids. Typically, the peptide linker has from 1-40 amino acids, such as from 1-30, such as from 1-20, such as from 1-10 amino acids. In a further embodiment the peptide linker is selected from a linker made up of amino acids selected from glycine, alanine, proline, asparagine, glutamine, and lysine. Typically, the peptide linker is made up of a majority of amino acids that are sterically unhindered, such as glycine and alanine. In particular, the peptide linker comprises a sequence selected from -(G)n-, (GGS)n or (GGGGS)n, wherein n is an integer of from 1-50. Typically n is an integer selected from 1-10, such as 1, 2, 3, 4, 5, 6, 7, 8, 9, or 10.

The antibody, antibody fragment, albumin, fatty acid or any other one of the half-life extending can be conjugated to SorCS1 via any suitable linker or linker region. The linker may be a disulphide bridge, such as a —S—S— bond between two cysteine (Cys) amino acid residues in each of the SorCS1, and the pharmaceutically acceptable molecule. The linker may be a fused linker meaning that SorCS1 can be expressed in a living cell as one polypeptide or protein. The linker may be a hydrophilic linker that separates an SorCS1 and a pharmaceutically acceptable molecule with a chemical moiety, which comprises at least 5 non-hydrogen atoms where 30-50% of these are either N or O. The linker may be hydrolysable as described in U.S. Pat. No. 6,515,100, U.S. Pat. No. 7,122,189, U.S. Pat. No. 7,700,551, WO2004/089280, WO2006/138572 and WO2009/095479. Typical compounds useful as linkers in the present invention include those selected from the group having dicarboxylic acids, malemido hydrazides, PDPH, SPDP, LC-SPDP, GMBS, carboxylic acid hydrazides, and small peptides. More specific examples of compounds useful as linkers, according to the present invention, include: (a) dicarboxylic acids such as succinic acid, glutaric acid, and adipic acid; (b) maleimido hydrazides such as N-[maleimidocaproic acid]hydrazide (EMCH), N-[maleimidopropionic acid]hydrazide (MPH or BMPH), 4-[N-maleimidomethyl]cyclohexan-1-carboxylhydrazide, and N-[k-maleimidoundcanoic acid]hydrazide (KMUH), 4-(4-N-MaleimidoPhenyl)butyric acid Hydrazide (MPBH); (c) NHS-3-maleimidopropionate Succinimide ester (MPS-EDA); (d) PDPH linkers such as (3-[2-pyridyldithio]propionyl hydrazide) conjugated to sulfurhydryl reactive protein; (e)N-Succinimidyl 3-(2-pyridyldithio)-propionate (SPDP), (f) Succinimidyl 6-(3-[2-pyridyldithio]-propionamido)hexanoate (LC-SPDP), (g)N-(v-Maleimidobutyryloxy)succinimide ester (GMBS), and (h) carboxylic acid hydrazides selected from 2-5 carbon atoms. Other non-peptide linkers are also possible. For example, alkyl linkers such as —NH—(CH2)m-C(0)-, wherein m is an integer selected from 2-20, could be used. These alkyl linkers may further be substituted by any non-sterically hindering group such as lower alkyl (e.g., C1 to C6) lower acyl, halogen (e.g., CI, Br, I, F), CN, NH2, phenyl, etc. An exemplary non-peptide linker is a PEG linker. Additional linkers useful according to the present invention are described in U.S. Pat. No. 6,660,843.

Different techniques for linking two or more molecules together, such as SorCS1 and the pharmaceutically acceptable molecule, and optionally via a multifunctional linker, such as bifunctional linker, are available in the prior art, and a suitable reference here is WO01/58493, including all relevant documents listed and cited therein.

In the present context, the term “a pharmaceutically acceptable molecule” as used herein means a molecule selected from any one of small organic molecules, peptides, oligopeptides, polypeptides, proteins, receptors, glycosylations, sugars, polymers (e.g. polyethylene glycols, PEG), nucleic acids (e.g. DNA and RNA), hormones, which when linked to SorCS1, increases the serum half-life of the SorCS1 or variant thereof. Typically, pharmaceutically acceptable molecules are without limitation albumin, such as human albumin, recombinant albumin, or polymer, such as PEG, e.g. PEG of a molecular weight of at least 10 kDa, such as from 10 kDa to 150 kDa. Furthermore, pharmaceutically acceptable molecules may be selected from a Fc fragment of a mammalian antibody, transferrin, albumin, such as human albumin, recombinant albumin, variants of albumin, CH₃(CH₂)ₙCO—, wherein n is 8 to 22, or polymer, such as PEG, e.g. PEG of a molecular weight of at least 5 kDa, such as from 10 kDa to 150 kDa, typically 10 to 40 kDa.

In the present context, the term “in vivo plasma half-life” is used in its normal meaning, i.e., the time required for the amount of SorCS1, in a biological system to be reduced to one half of its value by biological processes.

The term “serum half-life”, which may be used interchangeably with “plasma half-life” or “half-life” is used in its normal meaning, i.e., the time required for the amount of SorCS1 in a biological system to be reduced to one half of its concentration. Thus as used herein, the “serum half-life” means the serum half-life in vivo. Determination of serum half-life is often more simple than determining functional half-life and the magnitude of serum half-life is usually a good indication of the magnitude of functional in vivo half-life. Preferably the serum half-life is measured in a mammal, more preferably in a species of Hominidae, such as Orangutan, Chimpanzee or Gorillas, more preferably in humans. The serum half-lives mentioned in the present application are half-lives as determined in humans. An indication of the half-life or any change in half-life can also be obtained in rodents, such as mouse or rat or hamster. Furthermore half-life can be measured in larger mammals having a body weight in the same range as human beings or closer to human being body weight than rodents: preferably monkey, dog, pig, or cattle (calf).

The term “increased” as used in connection with the plasma half-life is used to indicate that the relevant half-life of the SorCS1 compound, as determined under comparable conditions. For instance the relevant half-life may be increased by at least about 25%, such as by at least about 50%, e.g., by at least about 100%, 150%, 200%, 250%, or 500%. Measurement of in vivo plasma half-life can be carried out in a number of ways as described in the literature. An increase in in-vivo plasma half-life may be quantified as a decrease in clearance or as an increase in mean residence time (MRT). The SorCS1 compound of the present invention for which the clearance is decreased to less than 70%, such as less than 50%, such as less than 20%, such as less than 10% of the clearance of the SorCS1, as determined in a suitable assay is said to have an increased in-vivo plasma half-life. SorCS1 of the present invention for which MRT is increased to more than 130%, such as more than 150%, such as more than 200%, such as more than 500% of the MRT of SorCS1, in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable test animals include normal, Sprague-Dawley male rats, mice and cynomolgus monkeys. Typically the mice and rats are injected in a single subcutaneous bolus, while monkeys may be injected in a single subcutaneous bolus or in a single iv dose. The amount injected depends on the test animal. Subsequently, blood samples are taken over a period of one to ten days as appropriate (depending on the sensitivity of the assay it may be as long as 30 days) for the assessment of clearance and MRT. The blood samples are conveniently analysed by ELISA techniques or other immunological techniques.

In the present context, the term “plasma concentration” as used herein means the concentration that can be measured in circulation at any given time after injection of SorCS1. In the present context, the term “an injection” as used herein means administration by the parenteral route such as by subcutaneous, intramuscular, intraperitoneal or intravenous injection by means of a syringe or other administration device.

The most abundant protein component in circulating blood of mammalian species is serum albumin, which is normally present at a concentration of approximately 3 to 4.5 grams per 100 millilitres of whole blood. Serum albumin is a blood protein of approximately 70,000 Dalton (Da) which has several important functions in the circulatory system. It functions as a transporter of a variety of organic molecules found in the blood, as the main transporter of various metabolites such as fatty acids and bilirubin through the blood, and, owing to its abundance, as an osmotic regulator of the circulating blood. In the present context, the term “an albumin” as used herein means albumin of mammalian origin or non-mammalian origin, such as human serum albumin that is described in Peters, T., Jr. (1996) All about Albumin: Biochemistry, Genetics and Medical, Applications pp10, Academic Press, Inc., Orlando (ISBN O-12-5521 10-3), or recombinant human albumin, or modified albumin, such as human albumin modified as described in WO2011051489 and WO2010092135. WO2011051489 the specification relates to variants of a parent albumin having altered plasma half-life compared with the parent albumin. The present invention also relates to fusion polypeptides and conjugates comprising said variant albumin.

WO2010092135 based on the three-dimensional structure of albumin, the inventors have designed variant polypeptides (muteins) which have one or more cysteine residues with a free thiol group (hereinafter referred to as “thio-albumin”). The variant polypeptide may be conjugated through the sulphur atom of the cysteine residue to a conjugation partner such as a bioactive compound.

WO2005054286 the specification relates to proteins comprising Interleukin 11 (IL-11) (including, but not limited to, fragments and variants thereof), which exhibit thrombopoietic or antiinflammatory properties, fused to albumin (including, but not limited to fragments or variants of albumin).

WO2004083245 describes an agent having a greater half-life than naturally produced albumin in a patient with MS, the agent comprising an albumin-like first polypeptide bound to a second polypeptide.

WO03066681 describes a composition comprising a non-albumin protein stabilised by the addition of a highly purified recombinant human serum albumin. The non-albumin protein may be Factor VIII.

In a further aspect the present invention relates to a method of preparing a long acting biologically active SorCS1 compound, such as any one of the herein disclosed conjugates of the present invention, comprising a SorCS1 polypeptide linked to a pharmaceutically acceptable molecule, the method comprising reacting a SorCS1 with a linker attached to a pharmaceutically acceptable molecule, or reacting a SorCS1 polypeptide with a linker and then attaching said linker to a pharmaceutically acceptable molecule, or reacting a linker with a pharmaceutically acceptable molecule and then reacting a SorCS1 polypeptide with the linker attached to the pharmaceutically acceptable molecule, or by expressing the SorCS1 polypeptide and the pharmaceutically acceptable molecule from a host cell.

In one embodiment the present invention relates to a long-acting modified mammalian SorCS1, e.g. human SorCS1 linked to such as fused to albumin, or conjugated to an acylation group or PEG and provides an in vivo plasma half-life of the mammalian SorCS1 or analog thereof, or the modified SorCS1 polypeptide which is from 2 to 48 hours in a mammal. The modified long acting SorCS1 is believed to improve patient convenience and treatment outcome by reducing the frequency of SorCS1 administration.

In another embodiment, increased halflife is obtained by use of a sustained delivery system or slow release delivery. For example, liposomes are well-known drug carriers, which could be employed for delivery of polypeptides of the present invention. In this case, liposomes could be produced, which comprise a SorCS1 polypeptide of the invention. Sustained delivery systems based on the biodegradable polymers poly(lactic acid) (PLA) and poly(lactic/glycolic acid) (PLGA) are also suitable for delivery of polypeptide drugs of the present invention.

In one embodiment the agent of the invention is modified in order to increase its half-life when administered to a patient, in particular its plasma half-life. The modification may be in the form of a moiety conjugated to the agent of the invention, thus generating a moiety-conjugated agent, wherein said moiety-conjugated agent has a plasma and/or serum half-life being longer than the plasma and/or serum half-life of the non-moiety conjugated agent. In one such embodiment the moiety conjugated to the agent is one or more type of moieties selected from the group consisting of albumin and variants thereof, fatty acids, polyethylene glycol (PEG), acylation groups, antibodies and antibody fragments. The conjugation of the moiety to the polypeptide of the invention may be to any suitable amino acid residue (backbone or side chain) of the polypeptide of the invention. The moiety may also be conjugated to polypeptide of the invention by a linker. In certain embodiments said linker has a sequence selected from the group consisting of SEQ ID NO:67, 68, 69, 70 and 71.

In one embodiment the moiety conjugated to the polypeptide according to the present invention is a moiety which facilitates transport across the blood brain barrier (BBB). An example of such a cross-BBB transport facilitator is an antibody from a camelid species. Camelids such as dromedaries, camels, llamas, alpacas, vicuñas, and guanacos have single-chain antibodies capable of crossing the BBB. The person of skill in the art is aware of how to See Li et al (2012) FASEB J. (10):3969-79

### Nucleic Acid, Vectors and Host Cells

As mentioned herein above, the present invention also comprises nucleotides capable of encoding the polypeptide as defined herein above, such as wherein the encoded polypeptide has at least 60%, e.g. 65%, e.g. 70%, e.g. 75%, such as 80%, e.g. 85%, such as 90%, e.g. 95%, such as 98%, e.g. 99% sequence identity to a sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 and 54 or to a fragment thereof.

In one aspect the invention relates to a vector, said vector comprising at least one nucleotide as defined herein above, for use in a method of reducing appetite in an individual.

In another aspect the invention relates to a vector, said vector comprising at least one nucleotide as defined herein above, for use in a method for promoting weight loss.

In another aspect the invention relates to a vector, said vector comprising at least one nucleotide as defined herein above, for use in a method for treating obesity.

In another aspect the invention relates to a vector, said vector comprising at least one nucleotide as defined herein above, for use in a method for increasing metabolism.

In another aspect the invention relates to a vector, said vector comprising at least one nucleotide as defined herein above, for use in a method for increasing thermogenesis.

In another aspect the invention relates to a vector, said vector comprising at least one nucleotide as defined herein above, for use in an in vivo and/or an in vitro method for converting white fat into brown fat.

The vector of the invention may further comprise a promoter which may be operably linked to the nucleic acid molecule of the invention. The promoter may be selected from, but is not limited to the group consisting of: CMV, human UbiC, RSV, Tet-regulatable promoter, Mo-MLV-LTR, Mx1, EF-1alpha, PDGF beta and CaMK II. The vector of the invention may also be selected from the group consisting of vectors derived from the Retroviridae family including lentivirus, HIV, SIV, FIV, EAIV, CIV. Other vectors of the invention are selected from the group consisting of adeno associated virus, adenovirus, alphavirus, baculovirus, HSV, coronavirus, Bovine papilloma virus, Mo-MLV, preferably adeno associated virus.

In another embodiment, the invention relates to a host cell comprising the nucleic acid as described above, wherein the isolated host cell is transformed or transduced with at least one vector as defined herein above. Thus the host cell may be implanted naked or in a biocompatible capsule thus producing the polypeptide of the present invention.

In one aspect the invention relates to a host cell comprising at least one nucleotide as defined herein above, for use in a method of reducing appetite in an individual.

In another aspect the invention relates to a host cell comprising at least one nucleotide as defined herein above, for use in a method for promoting weight loss.

In another aspect the invention relates to a host cell comprising at least one nucleotide as defined herein above, for use in a method for treating obesity.

In another aspect the invention relates to a host cell comprising at least one nucleotide as defined herein above, for use in a method for increasing metabolism.

In another aspect the invention relates to a host cell comprising at least one nucleotide as defined herein above, for use in a method for increasing thermogenesis.

In another aspect the invention relates to a host cell comprising at least one nucleotide as defined herein above, for use in an in vivo and/or an in vitro method for converting white fat into brown fat.

The isolated host may be selected from the group consisting of Saccharomyces cerevisiae, E. coli, Aspergillus and Sf9 insect cells and of mammalian cells selected from the group consisting of human, feline, porcine, simian, canine, murine and rat cells, wherein the mammalian cell may be selected from, but is not limited to the group consisting of muscle cells, hepatocytes, adipocytes and cells of the pancreas such as α cells, β cells and δ cells.

In one embodiment the isolated host cell is selected from the group consisting of CHO, CHO-K1, HEI193T, HEK293, COS, PC12, HiB5, RN33b and BHK cells.

In one embodiment the host cell is a human stem cell, and in another embodiment the host cell is not a human stem cell.

As discussed above the agent of the invention is any agent having the biological activity as demonstrated in the examples for soluble SorCS1 in relation to reducing appetite, and/or supressing hunger and/or reducing prospective consumption, and/or for promoting weight loss, and/or for treating obesity, and/or for increasing metabolism, and/or for increasing thermogenesis in a mammal, and/or for converting white fat into brown fat in vivo or in an in vitro cell culture. While it is preferred that the agent is a polypeptide, the agent may in principle be any type of molecule exhibiting the same biological response as a SorCS1 polypeptide, such as other polypeptides, in particular other Vps10p-domain receptors, antibodies as well as small organic molecules, wherein the antibody may be selected from the group consisting of: polyclonal antibodies, monoclonal antibodies, humanised antibodies, single chain antibodies and recombinant antibodies.

Furthermore, as discussed herein administration of nucleic acids either naked, or in host cells or packaging cells, wherein the nucleic acid is capable of encoding the SorCS1 polypeptide(s) as discussed herein, for the reduction of appetite, supression of hunger or reduction of desire to eat, is also an aspect of the invention.

### Methods of Screening for Agents of the Invention

The present invention provides specific targets and methods for screening and evaluating further candidate agents including SorCS1 peptide and polypeptide fragments and mutant and variants thereof.

While the screening of a large number of peptides for a certain physiological activity may be a laborious undertaking, the exact disclosures of the assay herein to be carried out enables the skilled person to reproduce the present invention without undue burden of experimentation and without needing inventive skill.

For this purpose screening libraries of candidate agents are readily available for purchase on the market. Whether a library is a peptide library or a chemical library does not have any impact in the present situation since screening of chemical libraries is also routine work. In fact screening of chemical libraries is a service offered by commercial companies, and it is clear from their presentation material that they do not consider the screening work as such to be inventive.

Initially in the process of screening for SorCS1-like agents i.e. agents exhibit the same biological response as SorCS1 such as reduction of appetite, promotion of weight loss, treatment of obesity, increased metabolism, increased thermogenesis, and/or conversion of white fat into brown fat, it is relevant to perform studies as discussed herein to verify that the agent is biologically active. As herein, this may be done indirectly by showing that administration of the SorCS1-like agent in fact results in a reduced appetite in a test model such as a mouse.

Accordingly, in one embodiment the present invention relates to an in vivo and/or in vitro method for screening for the ability of the SorCS1-like agent as defined herein above to reduce appetite, promote weight loss, treat obesity, increase metabolism, increase thermogenesis, and/or convert white fat into brown fat,

### Pharmaceutical Composition and Administration Forms

The present invention also encompasses pharmaceutical compositions comprising the agent as defined herein. In the present context the term agent and compound is considered synonyms when discussing the pharmaceutical composition.

In the present context, the term “a pharmaceutical composition” as used herein typically means a composition containing SorCS1 and/or a SorCS1 variant of the present invention, and optionally one or more pharmaceutically acceptable carriers or excipients, and may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications. Typically, the pharmaceutical compositions of the present invention may be formulated for parenteral administration e.g., by i.v. or subcutaneous injection, and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water. Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. The parenteral formulations typically will contain from about 0.0001 to about 25%, such as from about 0.5 to about 25%, by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 0.000001 to about 15% by weight, such as from about 0.000001 to about 5% by weight or from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use.

The main route of drug delivery according to this invention is however parenteral in order to introduce the agent into the blood stream to ultimately target the relevant tissue.

The agent may also be administered to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth.

In a preferred embodiment the agent of the invention is administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, which is by intranasal and oral inhalation administration. Appropriate dosage forms for such administration, such as an aerosol formulation or a metered dose inhaler, may be prepared by conventional techniques.

In one embodiment the pharmaceutical composition according to the present invention is formulated for parenteral administration such as by injection.

In a further embodiment the pharmaceutical composition according to the present invention is formulated for intravenous, intramuscular, intraspinal, intraperitoneal, subcutaneous, a bolus or a continuous administration.

The rate and frequency of the administration may be determined by the physician from a case to case basis. In one embodiment the administration occurs at intervals of 30 minutes to 24 hours, such as at intervals of 1 to 6 hours.

The duration of the treatment may vary depending on severity of the condition. In one embodiment the duration of the treatment is from 6 to 72 hours. In chronic cases the duration of the treatment may be lifelong.

The dosage can be determined by the physician in charge based on the characteristics of the patient and the means and mode of administration. In one embodiment of the present invention, the dosage of the active ingredient of the pharmaceutical composition as defined herein above, is between 10 μg to 500 mg per kg body mass, such as between 20 μg and 400 mg, e.g. between 30 μg and 300 mg, such as between 40 μg and 200 mg, e.g. between 50 μg and 100 mg, such as between 60 μg and 90 μg, e.g. between 70 μg and 80 μg.

The dosage may be administered as a bolus administration or as a continuous administration. In relation to bolus administration the pharmaceutical composition may be administered at intervals of 30 minutes to 24 hours, such as at intervals of 1 to 6 hours. When the administration is continuous it is administered over an interval of time that normally is from 6 hours to 7 days. However, normally the dosage will be administered as a bolus 1-3 times per day.

### Formulations

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefore.

In one embodiment the pharmaceutical composition as defined herein above comprises a pharmaceutically acceptable carrier.

The agents of the present invention may be formulated into a wide variety dosage forms, suitable for the various administration forms discussed above.

The pharmaceutical compositions and dosage forms may comprise the agents of the invention or its pharmaceutically acceptable salt or a crystal form thereof as the active component.

Furthermore, the pharmaceutical compositions may comprises pharmaceutically acceptable carriers that can be either solid or liquid.

Solid form preparations are normally provided for oral or enteral administration, such as powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. Powders and tablets preferably contain from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilizing agents, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically for transdermal or transmucosal administration. Regions for topical administration include the skin surface and also mucous membrane tissues of the vagina, rectum, nose, mouth, and throat. Compositions for topical administration via the skin and mucous membranes should not give rise to signs of irritation, such as swelling or redness. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, such as a sublingual tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or colouring agents. Formulations suitable for topical administration in the mouth include lozenges comprising active agents in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or nonionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Transdermal delivery may be accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

The compounds of the present invention may also be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Pharmaceutically Acceptable Salts

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pH of the pharmaceutical composition may be any pH suitable for physiological purposes such as between pH 4 and pH 9, preferably between 5 and 8, more preferably around pH 7.

### Kit of Parts

In one aspect the present invention relates to a kit in parts comprising:
a pharmaceutical composition as defined herein above
a medical instrument or other means for administering the medicament
instructions on how to use the kit in parts.
optionally a second active ingredient as defined herein above

In a further embodiment the instrument as defined herein above is a so called insulin pen described in US Pat. Nos. 5,462,535, U.S. Pat. No. 5,999,323 and U.S. Pat. No. 5,984,906.

The second ingredient may be any suitable active ingredient normally administered to individuals suffering from obesity or overweight.

In a further aspect the invention relates to a pharmaceutical composition comprising the agent as defined above; or the isolated nucleic acid sequence as defined above; or the expression vector as defined above; or a composition of host cells as defined above; or a packaging cell line as defined above, or a combination thereof.

### EXAMPLES

### Example 1

### Gene Expression Profiling of Adipose Tissue from SorCS1 Knockout Mice by PCR Arrays

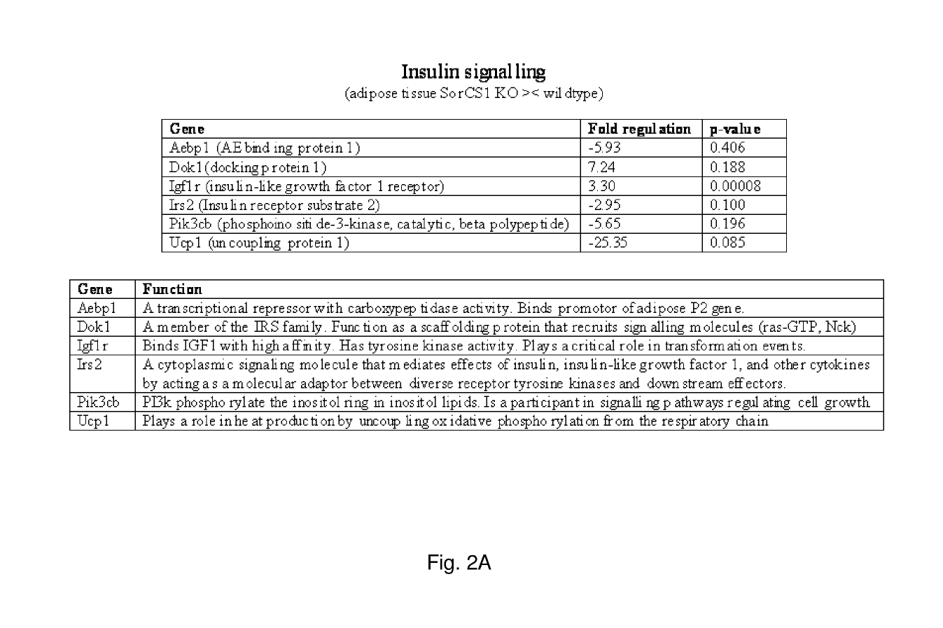

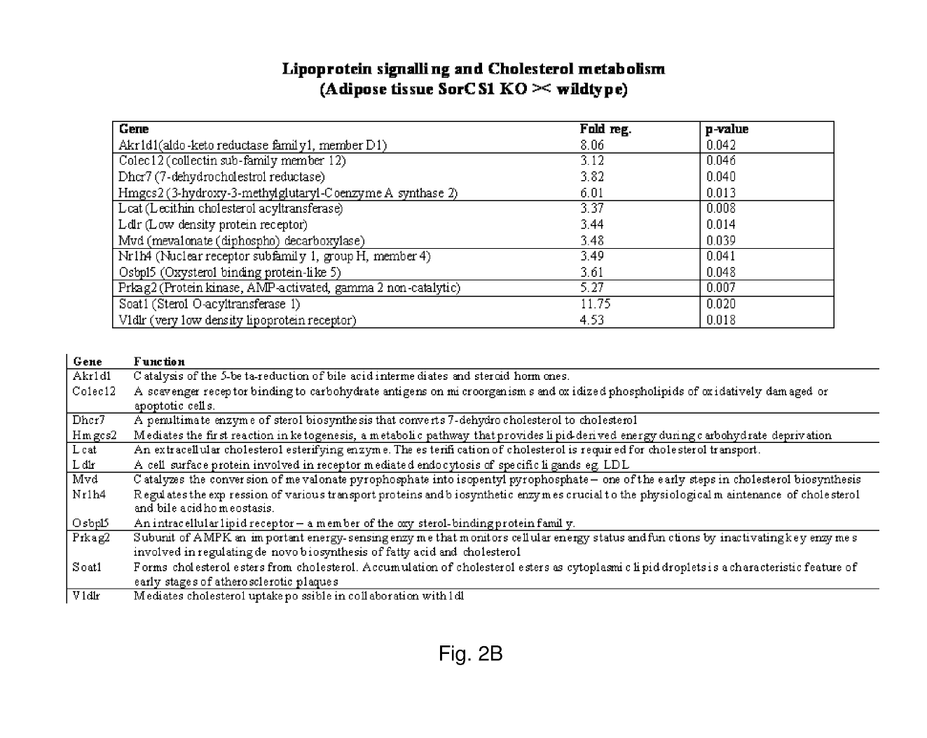

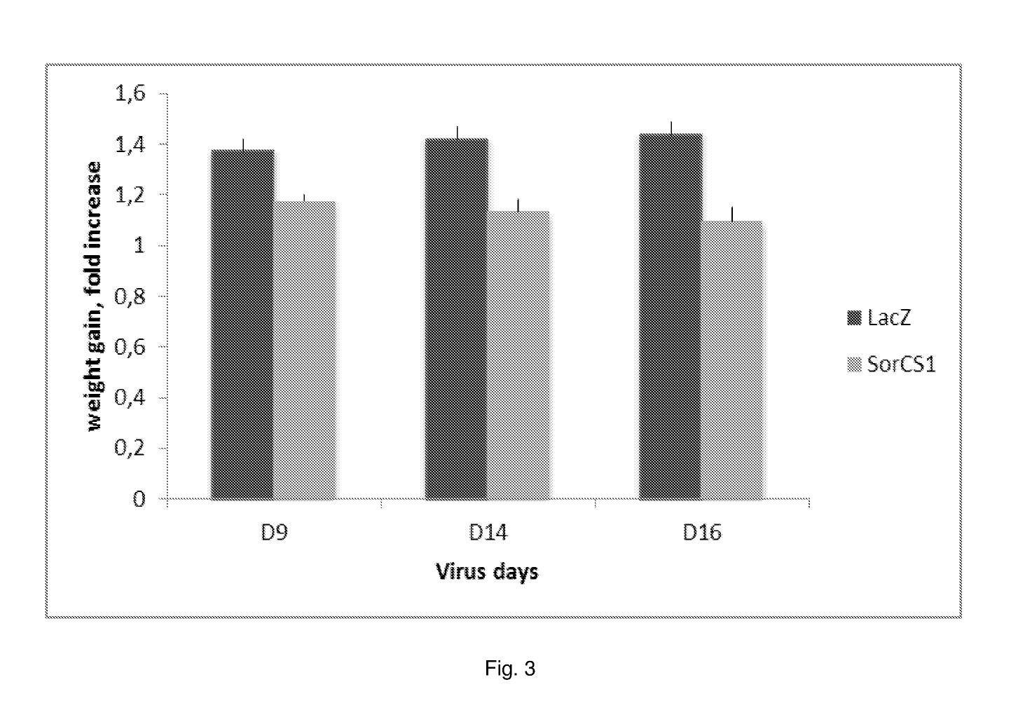

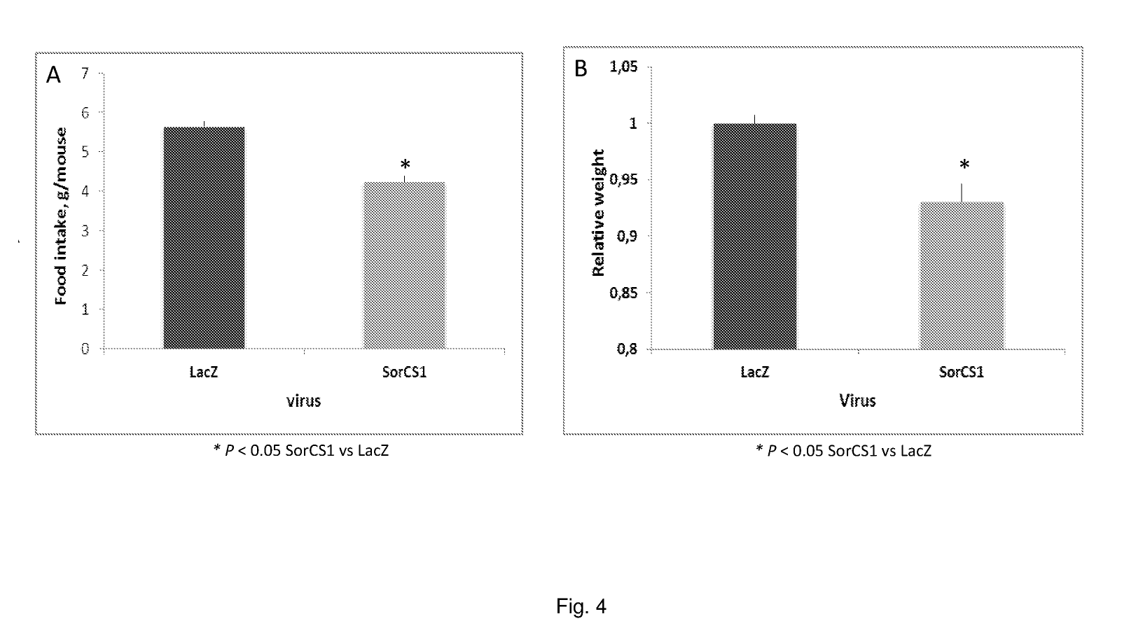

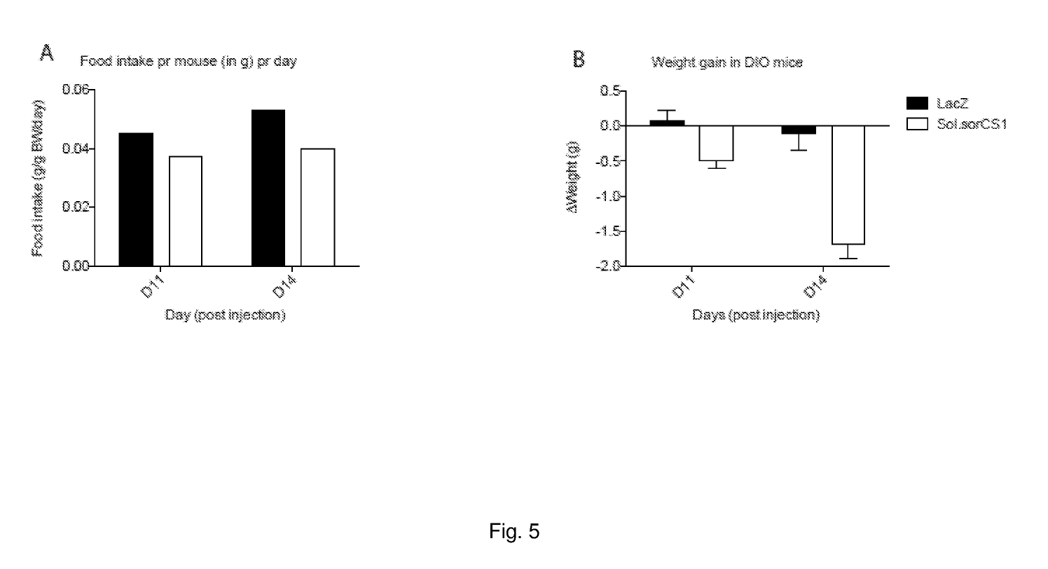

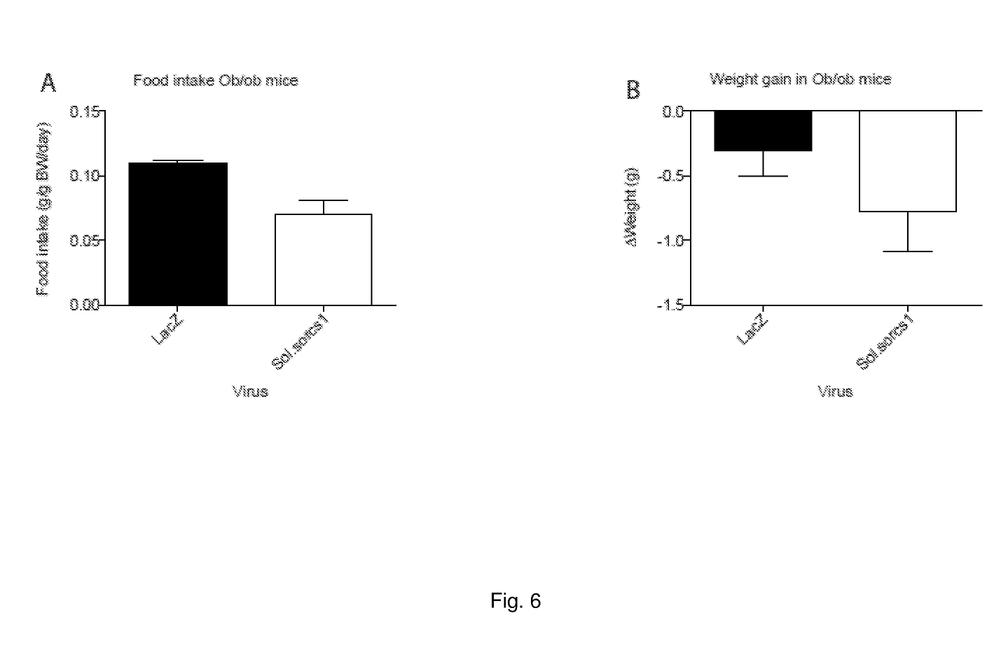

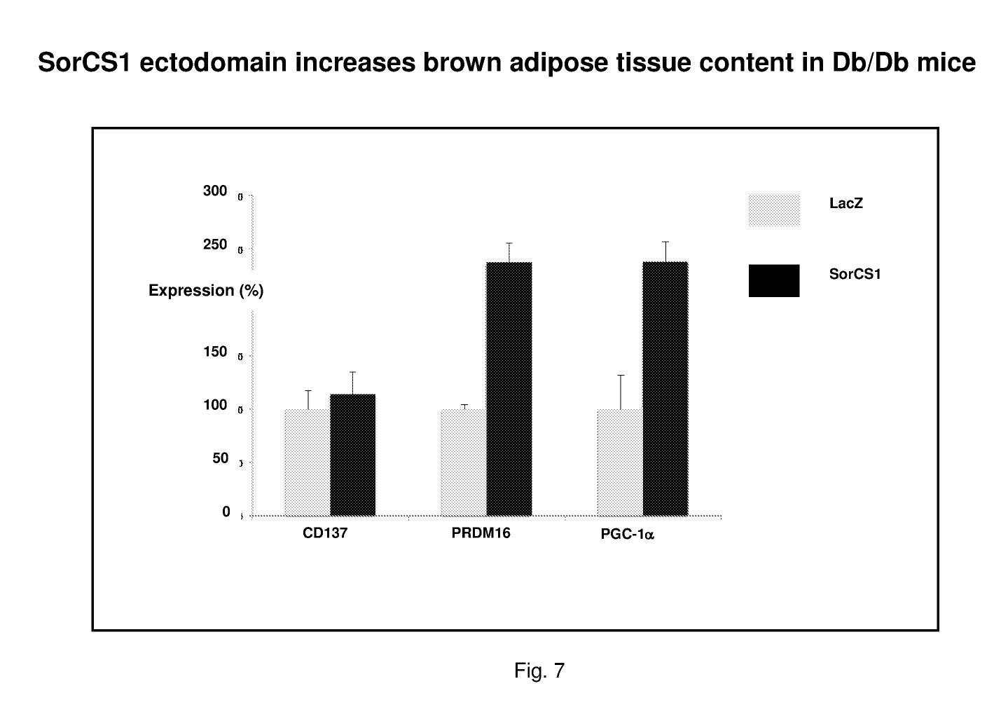

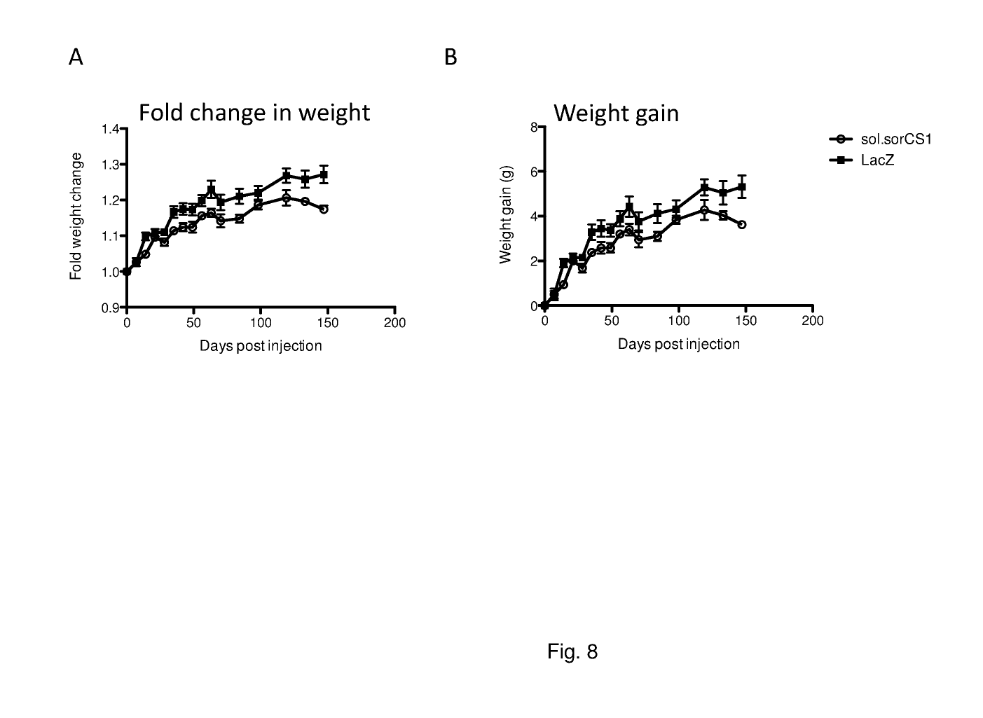

To examine the gene expression profile of SorCS1 knockout mice, the expression of 84 genes related to the mouse insulin signalling pathway and 84 genes related to mouse lipoprotein signalling & cholesterol metabolism was determined using microarray analysis. The microarray analysis was performed using RNA from adipose tissue of SorCS1 knockout wild-type adipose mice. In practice, first strand cDNA was synthesized from total RNA (Applied Biosystems) from SorCS1 knockout (−/−) and wild-type (+/+) adipose tissue from female mice 50 weeks of age (n=3). Then superarray of Mouse Insulin Signalling Pathway (PAMM-030A RT2 Profiler PCR arrays) or B) the type Mouse Lipoprotein Signalling & Cholesterol Metabolism (PAMM-080-A RT2 Profiler PCR arrays) were processed using an ABI7900 platform (Applied Biosystems) and SYBR Green/Rox PCR (SABiosciences). AROS Applied Biotechnology, Aarhus, Denmark, did the expression analyses. Genes showing an expression more than 3 times up- or down-regulated in the SorCS1 knockout mice when compared to wild-type mice are listed in the upper tables and their known functions are indicated in the table below. The data in FIG. 2A+B shows that the expression of several genes are changed in the SorCS1 knockout mice compared to the wild-type mice, indicating that insulin and cholesterol signalling pathways and metabolism are altered in SorCS1 knockout mice.

### Example 2

### Reduced Weight in Diabetic Db/Db Mice after Over-Expression of Soluble SorCS1

To evaluate the effect of soluble SorCS1 on weight in an obese mouse model that spontaneously develops type 2 diabetes, we used the db/db mouse strain (BKS.Cg-m+/+Lpr^{db}/BomTac from Taconic). These mice lack the leptin receptor and consequently the mice become obese and develop insulin resistance and finally severe diabetes at the age of 6-8 weeks.

We injected adenovirus expressing either human soluble (hsol.) SorCS1 or LacZ as a control, to examine the effect on weight. Recombinant adenovirus for expression of human soluble SorCS1 (hsol.SorCS1) was generated as follows: pcDNA3.1/Zeo(−)/hsol.SorCS1 encoding the human soluble SorCS1 cDNA (amino acids 1-1100) was digested with Pme1 and Apa1 and the fragment encoding hsol.SorCS1 inserted into the shuttle plasmid pVQpacAd5CMVK-NpA (ViraQuest Inc, North Liberty, Iowa). ViraQuest Inc, North Liberty, Iowa, then used this shuttle plasmid for generation and propagation of adenovirus over-expressing hsol. SorCS1. Adenovirus expressing LacZ as a negative control was obtained from ViraQuest Inc, North Liberty, Iowa

In detail, db/db female mice 6 weeks of age were injected in the tail vein with 2E9 pfu's of an adenoviral vector with either hsol.SorCS1 or LacZ (from ViraQuest Inc, North Liberty, Iowa) as a negative control virus. In the morning, on day 0, 9, 14 and day 16, the mice were weighed on a scale. Data are means±SEM for 5 mice in each group. On day 9 to 16, the db/db female mice with over-expression of soluble SorCS1 exhibited a significant decrease in weight compared to the mice that received the control LacZ virus. Thus, over-expression of soluble SorCS1 improves the obese status in this obese mouse model. The results are illustrated in FIG. 3

### Example 3

### Reduced Food Intake and Weight in Diabetic Db/Db Mice after Over-Expression of Soluble SorCS1

To evaluate the effect of soluble SorCS1 on weight in an obese mouse model that spontaneously develops type 2 diabetes, we used the db/db mouse strain (BKS.Cg-m+/+Lpr^{db}/BomTac from Taconic). These mice lack the leptin receptor and consequently the mice become obese and develop insulin resistance and finally severe diabetes at the age of 6-8 weeks. We injected adenovirus expressing either hsol.SorCS1 or LacZ as a control (see example 2), to examine the effect on weight. In detail, db/db female mice 6 weeks of age were injected in the tail vein with 2E9 pfu's of an adenoviral vector with either hsol.SorCS1 or LacZ (from ViraQuest Inc, North Liberty, Iowa) as a negative control virus. A) In the morning of day 9 after virus treatment each mouse was moved to a metabolic cage with a measured amount of food. 24 hours later the mouse was moved back to a normal mouse cage and the food in the metabolic cage was weighed to determine the food intake. The amount of ingested food over 24 hours is shown in FIG. 4A Data are means±SEM for 4 mice in each group. Mice with over-expression of soluble SorCS1 ate significant less than the control mice expressing LacZ. B) In the morning, on day 0 and 11 after virus treatment, the mice were weighed on a scale. The relative weight changes over the time period are shown. Data are means±SEM for 4 mice in each group. On day 11, the db/db female mice with over-expression of soluble SorCS1 exhibited a significant decrease in body weight compared to the mice that received the control LacZ virus. The results are illustrated in FIG. 4B

### Example 4

### Administering of Soluble SorCS1 or SorCS1 Peptides for the Treatment of Obesity

The soluble domain of mouse SorCS1 peptide(s) which is capable of binding to IR is expressed recombinantly in large scale, in a mammalian cell culture and is subsequently purified by for example immune-affinity chromatography. The protein or peptide is administered by peritoneal, intravenous, intramuscular or subcutaneous injection to e.g. an obese animal model (ob/ob or db/db mouse model) showing massive obesity (1 mg to 1 g/kg body weight each day or every week) in parallel with a wild type reference mouse. Good effect is obtained, and the same methods using human SorCS1 are applied for patients with obesity.

### Example 5

### Studies in Isolated Primary Adipocytes from Obese Mice

Primary cultures of adipocytes are isolated from obese mice (db/db or ob/ob) and treated with soluble SorCS1 or a control protein (delivered either as a virus or directly as a protein). Morphology and amount of adipokines are studied, and tested for ³H-glucose uptake in the different cell lines. Studies are undertaken of the insulin receptor and GLUT4 (stability, subcellular location, turnover), intracellular signaling cascades, and differentiation of primary cultures of adipocytes.

### Example 6

### Expression of Different Variants of SorCS1 in Human Adipose Tissue

Expression of SorCS1 polymorphisms and splice variants are investigated using quantitative PCR in adipose tissue from humans with obesity and/or type II diabetes.

### Example 7

### Fat Distribution in Obese Mice Treated with SorCS1 Using NMRI

Fat distribution is investigated in obese mice treated with either soluble SorCS1 or a control protein (delivered either as a virus or directly as a protein). The investigation is undertaken using NMR imaging (e.g. Siemens 3 Tesla or a custom-build 7 Tesla scanner available at the Department of Chemistry, Aarhus University, Denmark.

### Example 8

### Screening Assay for Identification of Active Polypeptides

The present assay is used to identify SorCS1 like agents having similar activity as the agents tested herein above. Such SorCS1 like agents include but is not limited to the other Vps10p-D receptors Sortilin (SEQ ID NO: 52), SorLA (SEQ ID NO: 53), SorCS2 (SEQ ID NO: 53) and SorCS3 (SEQ ID NO: 54).

Expression vectors containing nucleic acid sequences encoding candidate polypeptides such as fragments of sortilin, SorLA, SorCS2 and SorCS3 or other polypeptide and transfected into NIH 3T3-L1 mouse embryonic fibroblast cells. The pre-adipocyte 3T3-L1 cells differentiate into mature adipocytes when cultured in the presence of 0.5 M methylisobutylxanthine, 1 μM dexamethasone, 5 μg/ml insulin and 10% fetal bovine serum for 2 days. Cells are fed every 2 days with standard media without any additive for about 10 days. At that time, lipid droplets are visible by phase-contrast microscopy and the amount of the lipid droplets are measured and quantified to find the effect of the peptide on fat deposits and obesity development. Furthermore, Western Blot using antibodies against different differentiation markers, such as CCAAT/enhancer-binding proteins (C/EBPs) and peroxisome proliferator-activated receptors (PPARs), measures the effect of the different peptides on differentiation of the fibroblast into mature adipocytes.

### Example 9

### Investigation of Similar Effect of Other Vps10p Domain Receptors on Fat Distribution, Food Intake and Weight Development

To examine the effect of peptide fragments of sortilin, SorLA, SorCS2 and SorCS3 on weight gain and food intake, female db/db mice are injected with adenoviruses expressing either soluble peptide fragments of a candidate polypeptide such as sortilin/SorLA/SorCS2/SorCS3 or LacZ, as a control virus (see example 2 for generation of virus with soluble fragments). In detail, db/db female mice 6 weeks of age are injected in the tail vein with 2E9 pfu's of an adenoviral vector with either of the above mentioned VPS1 OP domain receptor fragments (which are found to have an effect on 3T3-L1 cells in example 8) or LacZ (from ViraQuest Inc, North Liberty, Iowa) as a negative control virus. In the morning of day 9 after virus treatment each mouse is moved to a metabolic cage with a measured amount of food. 24 hours later the mouse is moved back to a normal mouse cage and the food in the metabolic cage is weighed to determine the food intake. In the morning, on day 0 and 11 after virus treatment, the mice is weighed on a scale. The relative weight changes over the time period are measured. Fat distribution in the body in the mice is determined as described in example 7 using NMRI and an evaluation of the candidate polypeptide as a drug is performed.

### Example 10

### Reduced Food Intake and Weight in Obese DIO Male Mice after Over-Expression of Soluble SorCS1

To evaluate the effect of soluble SorCS1 on weight in an obese mouse model we used male mice 15 weeks of age from a diet-induced obesity (D10) mouse model (C57BL/6J DIO from Taconic). These mice have been placed on a 60 kcal % high fat diet from 6 weeks of age and as a consequence the mice become obese compared to mice on normal diet. We injected adenovirus expressing either hsol.SorCS1 or LacZ as a control to examine the effect on weight (see example 2 for virus details). In detail, DIO male mice 15 weeks of age were injected in the tail vein with 2E9 pfu's of an adenoviral vector with either hsol.SorCS1 or LacZ (from ViraQuest Inc, North Liberty, Iowa) as a negative control virus. A) In the morning of day 10 after virus treatment each group of virus treated mice were moved to a cage with a measured amount of food. Every 24 hours over the next 4 days the food in the cage were weighed to determine the food intake. The amount of eaten food over 24 hours is shown for day 11 and 14. Data are means±SEM for 5 mice in each group. Mice with over-expression of soluble SorCS1 ate significant less than the control mice expressing LacZ. The results are illustrated in FIG. 5A. B) In the morning, on day 0, 11 and 14 after virus treatment, the mice were weighed on a scale. The relative weight changes compared to day 0 over the time period are shown. Data are means±SEM for 5 mice in each group. On day 11 and 14, the DIO male mice with over-expression of soluble SorCS1 exhibited a significant decrease in weight compared to the mice that received the control LacZ virus. The results are illustrated in FIG. 5B.

### Example 11

### Reduced Food Intake and Weight in Obese and Diabetic Ob/Ob Female Mice after Over-Expression of Soluble SorCS1

To evaluate the effect of soluble SorCS1 on weight in an obese mouse model that spontaneously develops type 2 diabetes, we used the ob/ob mouse strain (B6.V-Lep^{ob}/J from Charles River). These mice lack the leptin protein so consequently the mice become obese and develop insulin resistance and finally severe diabetes at the age of 8-10 weeks. We injected adenovirus expressing either hsol.SorCS1 or LacZ as a control, to examine the effect on weight (for virus detail see example 2). In detail, ob/ob female mice 8 weeks of age were injected in the tail vein with 2E9 pfu's of an adenoviral vector with either hsol.SorCS1 or LacZ (from ViraQuest Inc, North Liberty, Iowa) as a negative control virus. A) In the morning of day 9 after virus treatment each mouse was moved to a metabolic cage with a measured amount of food. 24 hours later the mouse was moved back to a normal mouse cage and the food in the metabolic cage weight to determine the food intake. The amount of eaten food over 24 hours is shown. Data are means±SEM for 4 mice in each group. Mice with over-expression of soluble SorCS1 ate significant less than the control mice expressing LacZ. The results are illustrated in FIG. 6A. B) In the morning, on day 0 and 10 after virus treatment, the mice were weighed on a scale. The relative weight changes over the time period are shown. Data are means±SEM for 4 mice in each group. On day 10, the ob/ob female mice with over-expression of soluble SorCS1 exhibited a significant decrease in weight compared to the mice that received the control LacZ virus. The results are illustrated in FIG. 6B.

### Example 12

### Overexpression of Soluble SorCS1 by Adenovirus Increase Expression of PRDM16 and PGC-1Alpha in Adipose Tissue from Db/Db Mice

Db/db female mice 6 weeks of age were injected with 2E9 PFU/mouse of an adenovirus over-expressing soluble SorCS1 or an adenovirus over-expressing lacZ as a negative control (see example 2 for virus details). 14 days post injection gonadal adipose was harvested from the mice and subjected to quantitative RT-PCR (pPCR) to determine the expression of the specific fat genes CD137 (brite adipose tissue marker), PRDM16 and PGC-1α (brown adipose tissue marker) and GAPDH as a household gen. In detail, mRNA is isolated from adipose of db/db females injected with either hsol.SorCS1 (n=5) or lacZ (n=4) using the kit Nucleospin RNA/protein, (Macherey-Nagel). First strand cDNA was synthesized from the mRNA using a cDNA reverse transcription kit (Applied Biosystems) and then quantitative RT-PCR was performed as a TaqMan gene expression Assay (Applied Biosystems) with specific primers/probes for CD137 (Mm00441899_m1), PRDM16 (Mm00712556_m1), PGC-1α (Mm01208835-mM), GAPDH (Mm99999915_g1) (Applied Biosystems) using an Fluidigm Biomark system (48.48 chip). AROS Applied Biotechnology, Aarhus, Denmark, did the expression analyses. The array data were analyzed using the GAPDH data as internal control to normalize the sample data and it is found that mRNA from PRDM16 and PGC-1alpha are significant (p<0.05) more than 2-fold upregulated in the adipose tissue from db/db mice subjected to AV-sol.sorcs1 virus compared to the control db/db mice subjected to AV-lacZ virus. The statistical significance of difference in gene expression was assessed by student's t-test (2 tailed, 2 sample, equal variance). Several proteins are involved in the process of converting white adipose tissue (WAT) to brown adipose tissue (BAT) in mice, e.g. PRDM16 and PGC-1α. PRDM16 is selectively expressed in BAT, where it activates BAT-specific gene expression and represses WAT-specific gene expression, through an interaction with the co-receptor PGC-1α. Thus, the 2 fold up-regulation of both PRDM16 and PGC-1α in adipose tissue from db/db female mice injected with AV-sol.SorCS1 indicate that over-expression of soluble sorcs1 in the liver leads to conversion of WAT to BAT, and this could result in increased production of heat and finally less weight gain. The results are displayed in FIG. 7.

### Example 13

### Less Weight Gain in Animals, on Normal Chow (ND), Treated with Soluble SorCS1 Expressed by Adeno-Associated Virus

To evaluate the long-term effect of soluble SorCS1 on weight gain in a regular mouse, we used the C57BL6/j strain (C57BL6/j bom tac) (n=5-6 per group). The recombinant adeno-associated virus for expression of human soluble SorCS1 (hsol.SorCS1) was generated by ViraQuest (ViraQuest Inc, North Liberty, Iowa) as follows: pVQAd5CMVK-NpA/hsol.SorCS1 encoding the human soluble SorCS1 cDNA (amino acids 1-1100) was digested with SalI and the 3363 by fragment encoding hsol.SorCS1 was inserted into an AAV8 plasmid (ViraQuest Inc, North Liberty, Iowa) generating AAV8/hsol.SorCS1. The plasmid pVQAd5CMVK-NpA/hsol.SorCS1 was sent to ViraQuest, that used this shuttle plasmid for subcloning, generation and propagation of adeno-associated virus over-expressing hsol.SorCS1. The virus AAV8/ntLacZ that over-express LacZ as a negative control was also purchased from ViraQuest.

The mice were i.v. injected with either soluble sorcs1 (AAV8-hsol.sorCS1) or LacZ (AAV8-LacZ) adeno-associated virus at the age of 8 weeks. The titer of virus injected was 1E11 vgc/mouse (vgc=viral genome copies). After 48 hours of quarantine, the animals were transferred back to their normal housing facilities and fed standard chow in the entire experimental period. Hereafter, the mice were weighed every fortnight in the following 22 weeks. The mice treated with AAV8-hsol.sorcs1 gain less weight in the period they are followed. The reduction in weight gain is 32% compared to their controls (LacZ treated animals). The gain in AAV8-hsol.sorcs1 group and the -LacZ group is 3.62±0.14 g and 5.32±0.50 g, respectively. Data are means±SEM. The effect of the AAV-hsol.sorcs1 virus, on weight gain, last up to 150 days post injection of the virus (p=0.0296, 2-way ANOVA, treatment). The results are displayed in FIG. 8.

### Example 14

### Method of Producing Long-Acting SorCS1

A long-acting SorCS1 agent may be produced by chemical conjugation of SorCS1 to human serum albumin or a variant of human serum albumin.

Chemical conjugation can be performed using a multitude of different reactions and linkers known in the art, including linkers with a high covalent stability and linkers with lower covalent stability having the potential of releasing the active component from the albumin molecule typically by hydrolysation of a labile chemical bond.

Especially suitable is coupling to the free cysteine residue on the albumin molecule (Cys 34), e.g. by methods described in WO2010092135, especially the methods using PDPH (3-(2-pyridyldithio) propionyl hydrazide) to link albumin to SorCS1 via a hydrazone link to SorCS1. In another aspect the method in WO2010092135 using EMCH ((3,3″-N-(ε-maleimidocaproic acid) hydrazide) to link albumin to SorCS1 via a hydrazone link to SorCS1 is used.

Suitable attachment groups on the SorCS1 molecule include reactions for coupling to the glycosylation moieties of the SorCS1 molecule. Coupling to the glycosylation moieties is preferred as these are expected not to have direct interaction with the SorCS1 receptor and thereby the coupling will not interfere with the function.

Yet another coupling technology is described by Neose (see eg US2004/0126838) using enzymatic glycoconjugation. This technology can be used to link e.g. albumin to SorCS1 using a suitable linker.

In the special case where chemical conjugation to the SorCS1 molecule strongly reduces the functional activity it will be preferable to use a labile linker that can release a functional SorCS1. It is preferable to attach only one albumin molecule per SorCS1 molecule.

In another instance the coupling of the SorCS1 and the albumin molecule can be performed by genetic fusion of the two molecules. Two different orientation possibilities exist:

### NH₂—Albumin-SorCS1-COOH

NH₂—SorCS1-albumin-COOH

Albumin or albumin variants can be produced as described in WO2010092135.

The SorCS1 and the albumin can be conjugated using the PDPH or EMCH chemistry as described in WO2010092135.

The biopotency of long-acting SorCS1 will be determined using established in vivo assays. Taking into account the bioavailability and kinectics of a long-acting SorCS1 compound, a way to measure the effect in mice would be to measure food intake (g/day/mouse), food preference tests, and changes in weight (weekly weighing of the mice), and weekly MRI scans (for fat and lean body mass).

Further the in vitro bioactivity of long-acting SorCS1 will be determined using standard cell assays. In cell cultures (e.g. 3T3, primary adipocytes or HEK293 cells) the long-acting SorCS1 will be added to the medium, and in lysates of the cells, we will determine expression of the a) insulin receptor, the b) phosphorylated insulin receptor (the activated form), and c) GLUT4 (facilitates glucose influx in cells), and d) the localization of GLUT4 (cell membrane or vesicular) in biotinylation studies. In adipocytes (3T3 or primary adipocytes) we will also measure proteins relevant for transition from white adipose tissue (WAT) to brown adipose tissue (BAT) after addition of long-lasting SorCS1. Relevant proteins to measure could be UCP1, PRDM16 and PGC-alpha.

For all assays the bioactivity of long-acting SorCS1 will be compared to recombinant SorCS1 by using The National Institute of Biological Standards and Controls (NIBSC Herts, UK) appropriate standards.

The amount of SorCS1 protein in a given composition will be determined using standard immunological techniques such as ELISA assay or RIA assay and characterized by Western blotting and measurement of total protein content using Bradford and/or Lowry assays.

### Example 15

### Covalent Attachment of PEG to SorCS1

SorCS1 and variants thereof may be covalently linked to any suitable polyethylene (PEG) molecule such as but not limited to SPA-PEG 5000, SPA-PEG 12000 and SPA-PEG 20000 (NOF Corporation) as described below (“PEGylation of SorCS1 in solution”).

### PEGylation of SorCS1 in Solution

Human SorCS1 are PEGylated at a concentration of 250 μg/ml in 50 mM sodium phosphate, 100 mM NaCl, pH 8.5. The molar surplus of PEG is 5-100 times with respect to PEGylation sites on the protein. The reaction mixture is placed in a thermo mixer for 30 minutes at 37° C. at 1200 rpm. After 30 minutes, quenching of the reaction is obtained by adding a molar excess of glycine.

Cation exchange chromatography is applied to remove excess PEG, glycine and other byproducts from the reaction mixture. The PEGylation reaction mixture is diluted with 20 mM sodium citrate pH 2.5 until the ionic strength is less than 7 mS/cm. pH is adjusted to 2.5 using 5 N HCl. The mixture is applied to a SP-sepharose FF column equilibrated with 30 mM sodium citrate pH 2.5. Unbound material is washed off the column using 4 column volumes of equilibration buffer. PEGylated protein is eluted in three column volumes by adding 20 mM sodium citrate, 750 mM sodium chloride. Pure PEGylated SorCS1 is concentrated and buffer exchange is performed using VivaSpin concentration devices, molecular weight cut-off (MWCO): 10 kDa.

### REFERENCES

1. P. Zimmet et al. (2005) The metabolic syndrome: A global public health problem and a new definition. J. Arthero. Thromb. 12(6) pp. 295-300
2. K. Srinivasan and P. Ramarao (2007) Animal models in type 2 diabetes research: An overview. Indian J. Med. Res. 125, pp 451-472
3. L. Plum et al. (2004) Transgenic and knockout mice in diabetes research: Novel insights into pathophysiology, limitations, and perspectives. Physiology 20 pp. 152-61
4. P. C. Champe and R. A. Harvey (2005) Diabetes Mellitus. Biochemistry 3rd Chapter 25
5. M. A. Herman and B. B. Kahn (2006) Glucose transport and sensing in them maintenance of glucose homeostasis and metabolic harmony. J. Cli, Invest. 116 pp. 1767-75 Pharm. Res. 57 pp 6-18
6. S. Koren and G. Fantus (2007) Inhibition of the protein tyrosine phosphatase PTP1B: potential therapy for obesity, insulin resistance and type-2 diabetes mellitus. Prac. Res. Clin. Endo. Meta. 21(4) pp 621-640
7. J. C. Hou and J. E. Pessin (2007) Ins (endocytosis) and outs (exocytosis) of GLUT4 trafficking. Cur. Opin. Cell. Biol. 19 pp 466-473
8. T. E. Graham and B. B. Kahn (2007) Tissue-specific alterations of glucose transport and molecular mechanisms of intertissue communication in obesity and type 2 diabetes. Horm. Metab. Res. 39 pp 717-721
9. C. Guerra et al. (2001) Brown adipose tissue-specific insulin receptor knockout shows diabetic phenotype without insulin resistance. J. Clin. Invest. 108(8) pp 1205-1213
10. G. Hermey et al. (1999) Identification and characterization of SorCS, a third member of a novel receptor family. Biochem. Biophys. Res. Commun. 266(2) pp. 347-51
11. A. Nykjaer et al. (2004) Sortilin is essential for proNGF-induced neuronal death. Nature 427(6977) pp. 843-8
12. O. M. Andersen et al. (2005) Neuronal sorting protein-related receptor SorLA/LR11 regulates processing of the amyloid precursor protein. Proc. Natl. Acad. Sci. USA. 102(38) pp. 13461-13466
13. N. J. Morris et al. (1998) Sortilin is the major 110-kDa protein in GLUT4 vesicles from adipocytes. J. Biol. Chem. 273(6) pp. 3582-7
14. J. Shi and V. Kandror (2005) Sortilin is essential and sufficient for the formation of Glut4 storage vesicles in 3T3-L1 adipocytes. Dev. Cell 9 pp 99-108
15. G. Hermey and H. C. Schaller (2000) Alternative splicing of murine SorCS leads to two forms of the receptor that differ completely in their cytoplasmic tails. Biochim. Biophys. Acta. 1491(1-3) pp. 350-54
16. G. Hermey et al. (2003) Characterization of SorCS1, an alternatively spliced receptor with completely different cytoplasmic domains that mediate different trafficking in cells. J. Biol. Chem. 278 pp. 7390-96
17. M. S. Nielsen et al. (2008) Different motifs regulate trafficking of SorCS1 isoforms. Traffic 9 pp. 980-94
18. S. M. Clee et al. (2006) Positional of SorCS1, a type 2 diabetes quantitative trait locus. Nature genetics 6 pp. 688-93
19. M. O. Goodarzi et al. (2007) SorCS1: A novel human type 2 diabetes susceptibility gene suggested by the mouse. Diabetes 56(7) pp. 1922-9
20. WO 2004/022719 (Attie et al.)

### OVERVIEW OF SEQUENCES

SEQ ID NO 1: Homo sapiens preproSorCS1 b (Isoform 1)
SEQ ID NO 2: Homo sapiens preproSorCS1 (Isoform 2)
SEQ ID NO 3: Homo sapiens preproSorCS1c (Isoform 3)
SEQ ID NO 4: Homo sapiens preproSorCS1a (Isoform 4)
SEQ ID NO 5: Soluble Homo sapiens preproSorCS1
SEQ ID NO 6: Homo sapiens proSorCS1b (Isoform 1)
SEQ ID NO 7: Homo sapiens proSorCS1 (Isoform 2)
SEQ ID NO 8: Homo sapiens proSorCS1c (Isoform 3)
SEQ ID NO 9: Homo sapiens proSorCS1a (Isoform 4)
SEQ ID NO 10: Soluble Homo sapiens proSorCS1
SEQ ID NO 11: Homo sapiens mature SorCS1b (Isoform 1)
SEQ ID NO 12: Homo sapiens mature SorCS1 (Isoform 2)
SEQ ID NO 13: Homo sapiens mature SorCS1c (Isoform 3)
SEQ ID NO 14: Homo sapiens mature SorCS1a (Isoform 4)
SEQ ID NO 15: Soluble Homo sapiens mature SorCS1
SEQ ID NO 16: Mouse preproSorCS1b (isoform 1)
SEQ ID NO 17: Mouse preproSorCS1a (isoform 2)
SEQ ID NO 18: Mouse preproSorCS1c (isoform 3)
SEQ ID NO 19: Mouse preproSorCS1c+ (isoform 4)
SEQ ID NO 20: Mouse preproSorCS1d
SEQ ID NO 21: Soluble mouse preproSorCS1
SEQ ID NO 22: Mouse proSorCS1 b (isoform 1)
SEQ ID NO 23: Mouse proSorCS1a (isoform 2)
SEQ ID NO 24: Mouse proSorCS1c (isoform 3)
SEQ ID NO 25: Mouse proSorCS1c+ (isoform 4)
SEQ ID NO 26: Mouse proSorCS1d
SEQ ID NO 27: Soluble mouse proSorCS1
SEQ ID NO 28: Mouse mature SorCS1b (isoform 1)
SEQ ID NO 29: Mouse mature SorCS1a (isoform 2)
SEQ ID NO 30: Mouse mature SorCS1c (isoform 3)
SEQ ID NO 31: Mouse mature SorCS1c+ (isoform 4)
SEQ ID NO 32: Mouse mature SorCS1d
SEQ ID NO 33: Soluble mouse mature SorCS1
SEQ ID NO 34: Chimpanzee preproSorCS1
SEQ ID NO 35: Chimpanzee proSorCS1
SEQ ID NO 36: Chimpanzee mature SorCS1
SEQ ID NO 37: Chimpanzee soluble SorCS1
SEQ ID NO 38: Dog mature SorCS1
SEQ ID NO 39: Dog soluble SorCS1
SEQ ID NO 40: Cow preproSorCS1
SEQ ID NO 41: Cow proSorCS1
SEQ ID NO 42: Cow mature SorCS1
SEQ ID NO 43: Cow soluble SorCS1
SEQ ID NO 44: Rat preproSorSC1
SEQ ID NO 45: Rat proSorCS1
SEQ ID NO 46: Rat mature SorCS1
SEQ ID NO 47: Rat soluble SorCS1
SEQ ID NO 48: Chicken preproSorCS1
SEQ ID NO 49: Chicken proSorCS1
SEQ ID NO 50: Chicken mature SorCS1
SEQ ID NO 51: Chicken soluble SorCS1
SEQ ID NO 52: Homo sapiens preproSortilin
SEQ ID NO 53: Homo sapiens preproSorLA
SEQ ID NO 54: Homo sapiens preproSorCS2
SEQ ID NO 55: Homo sapiens preproSorCS3
SEQ ID NO 56: Homo sapiens proSorCS3
SEQ ID NO 57: Homo sapiens mature SorCS3
SEQ ID NO 58: Homo sapiens soluble preproSorCS3
SEQ ID NO 59: Homo sapiens soluble proSorCS3
SEQ ID NO 60: Homo sapiens soluble mature SorCS3
SEQ ID NO 61: Homo Sapiens proSorCS1B variant
SEQ ID NO 62: Homo Sapiens soluble proSorCS1B variant
SEQ ID NO 63: Homo Sapiens mature SorCS1B variant
SEQ ID NO 64: Homo Sapiens soluble mature SorCS1B variant

### SEQ ID NO 65: Linker—SGGSGGS

### SEQ ID NO 66: Linker—GGSGGSGGSGGSGGG

### SEQ ID NO 67: Linker—GGSGGSGGSGGSGGSGGS

### SEQ ID NO 68: Linker—GGGGSGGGGSGGGGS

SEQ ID NO 69: Linker—EFAGAAAV

1. An agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
for use in a method for reducing appetite, and/or for treating obesity, and/or for promoting weight loss, and/or increasing metabolism, and/or increasing thermogenesis, and/or converting white fat into brown fat.

2. The agent according to claim 1 wherein the agent is a polypeptide, wherein the polypeptide is a biologically active sequence variant comprising an amino acid sequence selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 55, 56, 57, 58, 59, 60, 61, 62, 63 and 64.

3. The agent according to claim 1 wherein the agent is a polypeptide, wherein the polypeptide is a biologically active sequence variant having an amino acid sequence selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 55, 56, 57, 58, 59, 60, 61, 62, 63 and 64.

4. The agent according to claim 1, wherein the polypeptide is a naturally occurring allelic variant of a sequence selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 55, 56, 57, 58, 59, 60, 61, 62, 63 and 64.

5. The agent according to claim 1, wherein the polypeptide comprises an amino acid sequence of a soluble SorCS1 selected from the group consisting of: SEQ ID NOs: 15, 5, 63, 62, 21, 27, 33, 37, 39, 43, 47, 51.

6. The agent according to any one of the preceding claims, wherein the polypeptide is a variant polypeptide described therein, wherein any amino acid specified in the selected sequence is altered to provide a conservative substitution, with the proviso that no more than 200 amino acids are so altered.

7. The agent according to any one of the preceding claims, wherein the polypeptide is a variant polypeptide described therein, wherein any amino acid specified in the selected sequence is altered to provide a conservative substitution, with the proviso that no more than 100 amino acids are so altered.

8. The agent according to any one of the preceding claims, wherein the polypeptide is a variant polypeptide described therein, wherein any amino acid specified in the selected sequence is altered to provide a conservative substitution, with the proviso that no more than 50 amino acids are so altered.

9. The agent according to any one of the preceding claims, wherein the polypeptide is a variant polypeptide described therein, wherein any amino acid specified in the selected sequence is altered to provide a conservative substitution, with the proviso that no more than 25 amino acids are so altered.

10. The agent according to any one of the preceding claims, wherein said polypeptide has at least 65%, more preferably at least 70%, more preferably at least 75%, preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% sequence identity to an amino acid sequence selected from the group consisting of SEQ ID NOs: 15, 5, 1, 2, 3, 4, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 55, 56, 57, 58, 59, 60, 61, 62, 63 and 64.

11. The agent according to any one of the preceding claims, wherein said agent is a polypeptide, wherein the polypeptide is selected from the group consisting of SEQ ID NOs: 1, 2, 3, 4, 6, 7, 8, 9, 11, 12, 13, 14, 61 and 62.

12. The agent according to any one of the preceding claims, wherein the polypeptide is selected from the group consisting of SEQ ID NOs: 16, 17, 18, 19, 20, 22, 26, 28, 29, 30, 31 and 32.

13. The agent according to any one of the preceding claims, wherein the agent is a polypeptide selected from the group consisting of SEQ ID NOs: 61, 62, 63 and 64.

14. The agent according to any one of the preceding claims, wherein the agent is a polypeptide selected from the group consisting of SEQ ID NOs: 55, 56, 57, 58, 59 and 60.

15. The agent according to any one of the preceding claims, wherein the agent is a polypeptide selected from the group consisting of SEQ ID NOs: 62 and 64.

16. The agent according to claim 1, wherein the agent is a biologically active fragment, wherein the fragment comprises less than 500 contiguous amino acid residues, such as less than 450 contiguous amino acid residues, for example less than 400 contiguous amino acid residues, such as less than 350 contiguous amino acid residues, for example less than 300 contiguous amino acid residues, for example less than 250 contiguous amino acid residues, such as less than 240 contiguous amino acid residues, for example less than 225 contiguous amino acid residues, such as less than 200 contiguous amino acid residues, for example less than 180 contiguous amino acid residues, such as less than 160 contiguous amino acid residues, for example less than 150 contiguous amino acid residues, such as less than 140 contiguous amino acid residues, for example less than 130 contiguous amino acid residues, such as less than 120 contiguous amino acid residues, for example less than 110 contiguous amino acid residues, such as less than 100 contiguous amino acid residues, for example less than 90 contiguous amino acid residues, such as less than 85 contiguous amino acid residues, for example less than 80 contiguous amino acid residues, such as less than 75 contiguous amino acid residues, for example less than 70 contiguous amino acid residues, such as less than 65 contiguous amino acid residues, for example less than 60 contiguous amino acid residues, such as less than 55 contiguous amino acid residues, for example less than 50 contiguous amino acid residues, such as less than 45 contiguous amino acid residues, for example less than 40 contiguous amino acid residues, such as 35 contiguous amino acid residues, for example 30 contiguous amino acid residues, such as 25 contiguous amino acid residues, such as 20 contiguous amino acid residues, for example 15 contiguous amino acid residues of an any one of the amino acid sequences selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64.

17. The agent according to claim 1, wherein the agent is a biologically active fragment, wherein the fragment comprises at least 15 contiguous amino acid residues, such as more than 20 contiguous amino acid residues, for example more than 25 contiguous amino acid residues, for example more than 50 contiguous amino acid residues, such as more than 75 contiguous amino acid residues, for example more than 100 contiguous amino acid residues, such as more than 125 contiguous amino acid residues, for example more than 150 contiguous amino acid residues, such as more than 175 contiguous amino acid residues, for example more than 200 contiguous amino acid residues, such as more than 225 contiguous amino acid residues, for example more than 250 contiguous amino acid residues, such as more than 275 contiguous amino acid residues, for example more than 300 contiguous amino acid residues, such as more than 325 contiguous amino acid residues, for example more than 350 contiguous amino acid residues, such as more than 375 contiguous amino acid residues, for example more than 400 contiguous amino acid residues, such as more than 425 contiguous amino acid residues, for example more than 450 contiguous amino acid residues, such as more than 475 contiguous amino acid residues, for example more than 500 contiguous amino acid residues, such as more than 525 contiguous amino acid residues, for example more than 550 contiguous amino acid residues, such as more than 575 contiguous amino acid residues, for example more than 600 contiguous amino acid residues, such as more than 625 contiguous amino acid residues, for example more than 650 contiguous amino acid residues, such as more than 675 contiguous amino acid residues, such as more than 700 contiguous amino acid residues of any one of the amino acid sequences selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63 and 64.

18. The agent according to any one of the preceding claims wherein the polypeptide is glycosylated.

19. The agent according to claim 18, wherein the polypeptide is N-glycosylated in one or more asparagin amino acid residues corresponding to amino acid positions 184, 352, 433, 765, 776, 816, 847, 908 and 929 of SEQ ID NO: 1.

20. The agent according to any one of the preceding claims, wherein the polypeptide comprises one of more of the following sequences:
SEQ ID NO: 1 aa 103-124
SEQ ID NO: 1 aa 125-143
SEQ ID NO: 1 aa 144-162
SEQ ID NO: 1 aa 197-218
SEQ ID NO: 1 aa 391-409
SEQ ID NO: 1 aa 661-684
SEQ ID NO: 1 aa 763-783
SEQ ID NO: 1 aa 859-876.

21. The polypeptide of claim 1, wherein the signal peptide has been replaced by a heterologous signal peptide.

22. The agent according to any one of the preceding claims, wherein the polypeptide is capable of forming at least one intramolecular cystin bridge.

23. The agent according any one of the preceding claims, comprising a dimer of said polypeptide linked through at least one intermolecular cystin bridge.

24. The agent according to any one of the preceding claims, wherein said polypeptide further comprises an affinity tag, such as a polyhis tag, a GST tag, a HA tag, a Flag tag, a C-myc tag, a HSV tag, a V5 tag, a maltose binding protein tag, a cellulose binding domain tag.

25. The agent according to claim 1, wherein the vector further comprises a promoter operably linked to the nucleic acid sequence.

26. The agent according to claim 25, wherein the promoter is selected from the group consisting of: CMV, human UbiC, RSV, Tet-regulatable promoter, Mo-MLV-LTR, Mx1, EF-1 alpha, PDGF beta and CaMK II.

27. The agent according to claim 1, wherein the vector is selected from the group consisting of vectors derived from the Retroviridae family including lentivirus, HIV, SIV, FIV, EAIV, CIV.

28. The agent according to claim 1, wherein the vector is selected from the group consisting of adeno associated virus, adenovirus, alphavirus, baculovirus, HSV, coronavirus, Bovine papilloma virus, Mo-MLV.

29. The agent according to claim 1, wherein the vector is adeno associated virus (AAV).

30. The agent according to claim 1, wherein the host cell is selected from the group consisting of Saccharomyces cerevisiae, E. coli, Aspergillus and insect cells such as Sf9 insect cells.

31. The agent according to claim 1, wherein the host cell is selected from the group consisting of mammalian cells selected from the group consisting of human, feline, porcine, simian, canine, murine and rat cells.

32. The agent according to claim 31 wherein said mammalian cell is selected from the group consisting of muscle cells, hepatocytes, adipocytes and cells of the pancreas such as a cells, 13 cells and 8 cells.

33. The agent according to claim 1, wherein said host cell is selected from the group consisting of CHO, CHO-K1, HEI193T, HEK293, COS, HiB5, RN33b and BHK cells.

34. The agent of claim 1, wherein said subject does not suffer from insulin resistance and/or diabetes mellitus type 2.

35. The agent according to any one of the preceding claims, wherein the agent is chemically modified in order to increase its half-life when administered to a patient, in particular its plasma half-life.

36. The agent according to any one of the preceding claims, wherein the agent is chemically modified in order to increase its half-life when administered to a patient, in particular its plasma half-life.

37. The agent according to any one of the preceding claims, wherein said agent further comprises a moiety conjugated to said agent, thus generating a moiety-conjugated agent

38. The agent according to claim 37, wherein the moiety-conjugated agent has a plasma and/or serum half-life being longer than the plasma and/or serum half-life of the non-moiety conjugated agent.

39. The agent according to claim 37, wherein the moiety facilitates transport across the blood brain barrier.

40. The agent according to claim 37, wherein the moiety is an antibody from a camelid species such as a recombinant or native single-chain antibody from dromedaries, camels, llamas, alpacas, vicuñas, or guanacos.

41. The agent according to any one of claims 37 to 40, wherein the moiety conjugated to the agent is one or more type of moieties selected from the group consisting of albumin, fatty acids, polyethylene glycol (PEG), acylation groups, antibodies and antibody fragments.

42. The agent according to any one of claims 37 to 41, wherein the agent and the moiety are conjugated to each-other by a linker.

43. The agent according to any one of claims 37 to 41, wherein the more than one moiety is conjugated to the agent.

44. The agent according to any one of claims 42 and 43, wherein the linker is a peptide having an amino acid sequence selected from the group consisting of SEQ ID NO: 65, 66, 67, 68 and 69.

45. A pharmaceutical composition comprising the agent of any one of the preceding claims.

46. The pharmaceutical composition according to claim 45 further comprising a pharmaceutically acceptable carrier.

47. The pharmaceutical composition according to any one of claims 45 and 46 wherein the pH of the composition is between pH 4 and pH 10.

48. The pharmaceutical composition according to any one of claims 45 to 47 wherein the composition is formulated for parenteral administration.

49. The pharmaceutical composition according to any one of claims 45 to 47 wherein the composition is formulated for oral administration.

50. The pharmaceutical composition according to claim 48 wherein the parenteral administration is by injection.

51. The pharmaceutical composition according to any one of claims 48 and 49, wherein the administration is intravenous, intramuscular, intraspinal, intraperitoneal, subcutaneous, a bolus or a continuous administration.

52. The pharmaceutical composition according to any one of claims 45 to 51, wherein the administration occurs at intervals of 30 minutes to 24 hours, such as at intervals of 1 to 6 hours, such as three times a day.

53. The pharmaceutical composition according to any one of claims 45 to 52, wherein the duration of the treatment is from 6 to 72 hours.

54. The pharmaceutical composition according to any one of claims 45 to 52, wherein the duration of the treatment is from 24 hours to 7 days.

55. The pharmaceutical composition according to any one of claims 45 to 52, wherein the duration of the treatment is from 4 days to 150 days.

56. The pharmaceutical composition according to any one of claims 45 to 52, wherein the duration of the treatment is lifelong.

57. The pharmaceutical composition according to any one of claims 35 to 43, wherein the dosage of the active ingredient is between 10 μg to 500 mg per kg body mass, such as from 50 μg to 250 mg per kg body mass.

58. A kit comprising the pharmaceutical composition according to any one of claims 45 to 57, and instructions for use.

59. Use of an agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
for the preparation of a medicament for reduction of appetite, and/or for promoting weight loss, and/or increasing metabolism, and/or increasing thermogenesis, and/or converting white fat into brown fat.

60. A method for reducing appetite, and/or for promoting weight loss, and/or for treating obesity, and/or for increasing metabolism, and/or for increasing thermogenesis, and/or for converting white fat into brown fat, the method comprising administering to an individual in need thereof a therapeutically effective amount of an agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

61. A method for treating obesity the method comprising administering to an individual in need thereof a therapeutically effective amount of an agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

62. A method for increasing metabolism, the method comprising administering to an individual in need thereof a therapeutically effective amount of an agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

63. A method for increasing thermogenesis in a mammal, the method comprising administering to the mammal a therapeutically effective amount of an agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

64. An in vivo method for converting white fat into brown fat, the method comprising administering to a mammal a therapeutically effective amount of an agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).

65. An in vitro method for converting white fat into brown fat, the method comprising contacting a cell with an effective amount of an agent selected from the group consisting of:
a) an isolated polypeptide comprising:
i) the amino acid sequence of SEQ ID NOs: 15; or
ii) a biologically active sequence variant of the amino acid sequence of i) wherein the variant has at least 60% sequence identity to said SEQ ID NO: 15,
iii) a biologically active fragment of at least 15 contiguous amino acids of any one of i) through ii), said fragment having at least 60% sequence identity to SEQ ID NO: 15 in a range of overlap of at least 15 amino acids,
b) a nucleic acid sequence encoding a polypeptide as defined in a);
c) a vector comprising the nucleic acid molecule as defined in b),
d) an isolated host cell transformed or transduced with the nucleic acid of b) or the vector of c).
